SEQUENCE LISTING (1 sequence)

SEQ ID NO: 1 - protein, 35 aa, Homo sapien
EPLPDCCRQKTCSCRLYELLHGAGNHAAGILTLNH

(19) United States
(12) Patent Application Publication
(10) Pub. No.: US 2004/0192673 A1
(43) Pub. Date: Sep. 30, 2004

(54) N-aroyl cyclic amine derivatives as orexin receptor antagonists

(76) Inventors: Pascale Gaillard, Harlow (GB); Christopher Norbert Johnson, Harlow (GB); Riccardo Novelli, Harlow (GB); Roderick Alan Porter, Harlow (GB); Geoffrey Stemp, Harlow (GB); Kevin Michael Thewlis, Harlow (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/476,995

(22) PCT Filed: May 3, 2002
(86) PCT No.: PCT/EP02/04874

(30) Foreign Application Priority Data
May 5, 2001 (GB) 0111183.0
Dec. 19, 2001 (GB) 0130386.6

(51) Int. Cl. C07D 413/02; C 07D 4 3/02; A61K 31/55

(52) U.S. Cl. 514/217.04; 514/217.08; 514/235.2; 514/326; 514/318; 540/597; 544/124; 544/141; 546/207; 546/192

(57) ABSTRACT

This invention relates to N-aroyl cyclic amine derivatives of formula (I): wherein: Y represents a bond, oxygen, or a group (CH₂)ₙ, wherein n represents 1, 2 or 3 m represents 1, 2 or 3; p represents 0 or 1; X is O,SC═O, SO₂, or —CH═CH; Ar¹ is aryl, or a mono or bicyclic heteroaryl group containing up to 4 heteroatoms selected from N, O and S; any of which may be optionally substituted; Ar² represents phenyl or a 5- or 5-membered heterocyclyl group containing up to 3 heteroatoms selected from N, O and S, wherein the phenyl or heterocyclyl group is substituted by R¹ and further optional substituents; or Ar² represents an optionally substituted bicyclic aromatic or bicyclic heteroaromatic group containing up to 3 heteroatoms selected from N, O and S; R¹ represents hydrogen, optionally substituted (C₁₋₄)alkoxy, halo, cyano, optionally substituted (C₁₋₁₆)alkyl, optionally substituted 5- or 6-membered heterocyclic ring containing up to 4 heteroatoms selected from N, O and S; or a pharmaceutical acceptable salt thereof; and their use as pharmaceuticals, specifically as orexin receptor antagonists.

### COMPOUNDS

This invention relates to N-aroyl cyclic amine derivatives and their use as pharmaceuticals.

Many medically significant biological processes are mediated by proteins participating in signal transduction pathways that involve G-proteins and/or second messengers.

Polypeptides and polynucleotides encoding the human 7-transmembrane G-protein coupled neuropeptide receptor, orexin-1 (HFGAN72), have been identified and are disclosed in EP-A-875565, EP-A-875566 and WO 96/34877. Polypeptides and polynucleotides encoding a second human orexin receptor, orexin-2 (HFGANP), have been identified and are disclosed in EP-A-893498.

Polypeptides and polynucleotides encoding polypeptides which are ligands for the orexin-1 receptor, e.g. orexin-A (Lig72A) are disclosed in EP-A-849361. Orexin receptors are found in the mammalian host and may be responsible for many biological functions, including pathologies including, but not limited to, depression; anxiety; addictions; obsessive compulsive disorder, affective neurosis/disorder; depressive neurosis/disorder; anxiety neurosis; dysthymic disorder; behaviour disorder; mood disorder; sexual dysfunction; psychosexual dysfunction; sex disorder; sexual disorder; schizophrenia; manic depression; delerium; dementia; severe mental retardation and dyskinesias such as Huntington's disease and Gilles de la Tourett's syndrome; disturbed biological and circadian rhythms; feeding disorders, such as anorexia, bulimia, cachexia, and obesity; diabetes; appetite/taste disorders; vomiting/nausea; asthma; cancer; Parkinson's disease; Cushing's syndrome/disease; basophil adenoma; prolactinoma; hyperprolactinemia; hypopituitarism; hypophysis tumor/adenoma; hypothalamic diseases; Froehlich's syndrome; adrenohypophysis disease; hypophysis disease; hypophysis tumor/adenoma; pituitary growth hormone; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; sleep disturbances associated with such diseases as neurological disorders, neuropathic pain and restless leg syndrome, heart and lung diseases; acute and congestive heart failure; hypotension; hypertension; urinary retention; osteoporosis; angina pectoris; myocardial infarction; ischaemic or haemorrhagic stroke; subarachnoid haemorrhage; head injury such as sub-arachnoid haemorrhage associated with traumatic head injury; ulcers; allergies; benign prostatic hypertrophy; chronic renal failure; renal disease; impaired glucose tolerance; migraine; hyperalgesia; pain; enhanced or exaggerated sensitivity to pain, such as hyperalgesia, causalgia and allodynia; acute pain; burn pain; atypical facial pain; neuropathic pain; back pain; complex regional pain syndromes I and II; ardtritic pain; sports injury pain; pain related to infection, e.g. HIV, post-polio syndrome, and post-herpetic neuralgia; phantom limb pain; labour pain; cancer pain; post-chemotherapy pain; post-stroke pain; post-operative pain; neuralgia; nausea, vomiting; conditions associated with visceral pain including irritable bowel syndrome, migraine and angina; urinary bladder incontinence e.g. urge incontinence; tolerance to narcotics or withdrawal from narcotics; sleep disorders; sleep apnea; narcolepsy; insomnia; parasomnia; jet-lag syndrome; and neurodegenerative disorders, which includes nosological entities such as disinhibition-dementia-parkinsonism-amyotrophy complex; pallido-ponto-nigral degeneration, epilepsy, and seizure disorders.

Experiments have shown that central administration of the ligand orexin-A (described in more detail below) stimulated food intake in freely-feeding rats during a 4 hour time period. This increase was approximately four-fold over control rats receiving vehicle. These data suggest that orexin-A may be an endogenous regulator of appetite. Therefore, antagonists of its receptor may be useful in the treatment of obesity and diabetes, see Cell, 1998, 92, 573-585.

There is a significant incidence of obesity in westernised societies. According to WHO definitions a mean of 35% of subjects in 39 studies were overweight and a further 22% clinically obese. It has been estimated that 5.7% of all healthcare costs in the USA are a consequence of obesity. About 85% of Type 2 diabetics are obese, and diet and exercise are of value in all diabetics. The incidence of diagnosed diabetes in westernised countries is typically 5% and there are estimated to be an equal number undiagnosed. The incidence of both diseases is rising, demonstrating the inadequacy of current treatments which may be either ineffective or have toxicity risks including cardiovascular effects. Treatment of diabetes with sulfonylureas or insulin can cause hypoglycaemia, whilst metformin causes GI side-effects. No drug treatment for Type 2 diabetes has been shown to reduce the long-term complications of the disease. Insulin sensitisers will be useful for many diabetics, however they do not have an anti-obesity effect.

Rat sleep/EEG studies have also shown that central administration of orexin-A, an agonist of the orexin receptors, causes a dose-related increase in arousal, largely at the expense of a reduction in paradoxical sleep and slow wave sleep 2, when administered at the onset of the normal sleep period. Therefore antagonists of its receptor may be useful in the treatment of sleep disorders including insomnia.

The present invention provides N-aroyl cyclic amine derivatives which are non-peptide antagonists of human orexin receptors, in particular orexin-1 receptors. In particular, these compounds are of potential use in the treatment of obesity, including obesity observed in Type 2 (non-insulin-dependent) diabetes patients, and/or sleep disorders, and/or stroke, particularly ischemic or haemorrhagic stroke, and/or for blocking the emetic response i.e. useful in the treatment of nausea and vomiting. International Patent Applications WO99/09024, WO99/58533, WO00/47577 and WO00/47580 disclose phenyl urea derivatives and WO00/47576 discloses quinolinyl cinnamide derivatives as orexin receptor antagonists. WO01/96302 discloses N-aroyl cyclic amine derivatives.

According to the invention there is provided a compound of formula (I):

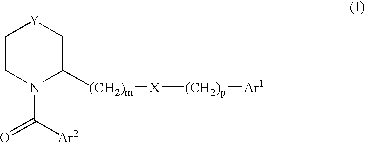

wherein:

Y represents a bond, oxygen, or a group (CH₂)ₙ, wherein n represents 1, 2 or 3

m represents 1, 2, or 3;

p represents 0 or 1;

X is O, S, C═O, SO₂, or —CH═CH—;

Ar¹ is aryl, or a mono or bicyclic heteroaryl group containing up to 4 heteroatoms selected from N, O and S; any of which may be optionally substituted;

Ar² represents phenyl or a 5- or 6-membered heterocyclyl group containing up to 3 heteroatoms selected from N, O and S, wherein the phenyl or heterocyclyl group is substituted by R¹ and further optional substituents; or Ar² represents an optionally substituted bicyclic aromatic or bicyclic heteroaromatic group containing up to 3 heteroatoms selected from N, O and S;

R¹ represents hydrogen, optionally substituted(C₁₋₄)alkoxy, halo, cyano, optionally substituted(C₁₋₆)alkyl, optionally substituted phenyl, or an optionally substituted 5- or 6-membered heterocyclic ring containing up to 4 heteroatoms selected from N, O and S;

or a pharmaceutically acceptable salt thereof.

Preferably where Ar² represents phenyl or a 5- or 6-membered heterocyclyl group containing up to 3 heteroatoms selected from N, O and S, the R¹ group is situated adjacent to the point of attachment to the amide carbonyl group, i.e. the R¹ is attached to Ar² in the ortho position to the amide carbonyl group.

When used herein the term amide carbonyl group means the —C(O)N— group as shown in compounds of formula (I).

Y is preferably a bond, oxygen or (CH₂)ₙ wherein n is 1 or 2.

X is preferably O, —CH═CH— or C═O.

m is preferably 1 or 2.

p is preferably 0.

Alternatively R¹ represents hydrogen, optionally substituted(C₁₋₄)alkoxy, halo, optionally substituted(C₁₋₆)alkyl, optionally substituted phenyl, or an optionally substituted 5- or 6-membered heterocyclic ring containing up to 3 heteroatoms selected from N, O and S.

Preferably R¹ represents an optionally substituted(C₁₋₄)alkoxy, halo, optionally substituted(C₁₋₆)alkyl, optionally substituted phenyl, or an optionally substituted 5- or 6-membered heterocyclic ring containing up to 3 heteroatoms selected from N, O and S.

More preferably R¹ is selected from trifluoromethoxy, methoxy, ethoxy, acetamido, halo, or an optionally substituted phenyl, pyridyl, pyrimidinyl, pyrazolyl or oxadiazolyl group.

Even more preferably R¹ is selected from trifluoromethoxy, methoxy, halo, or an optionally substituted phenyl, pyridyl, pyrazolyl or oxadiazolyl group.

Preferably Ar¹ is aryl, or a mono or bicyclic heteroaryl group containing up to 3 heteroatoms selected from N, O and S; any of which may be optionally substituted;

When Ar¹ is optionally substituted aryl it is preferably phenyl or naphthyl. The aryl group may have up to 5, preferably 1, 2 or 3 optional substituents.

When Ar¹ is a mono or bicyclic heteroaryl it is for example quinoxalinyl, quinazolinyl, pyridopyrazinyl, benzoxazolyl, benzothienyl, benzimidazolyl, naphthyridinyl, pyridinyl, pyrimidinyl or thiazolyl. Additionally it may be quinolinyl, isoquinolinyl, benzofuranyl, benzothiazolyl or indolyl. Furthermore it can be imidazolyl, oxazolyl, pyrazinyl, pyridazyl, thienyl, furanyl, oxadiazolyl or thiadiazolyl.

Preferably Ar¹ is phenyl, naphthyl, pyridinyl or benzofuranyl, more preferably pyridinyl or benzofuranyl. Even more preferably Ar¹ is pyridinyl.

When Ar² or R¹ is a 5- or 6-membered heterocyclyl group containing up to 4 heteroatoms selected from N, O and S, it may be furanyl, thienyl, pyrrolyl, oxazolyl, thiazolyl, inidazolyl, oxadiazolyl, thiadiazolyl, pyridyl, triazolyl, triazinyl, pyridazyl, pyrimidinyl, isothiazolyl, isoxazolyl, pyrazinyl or pyrazolyl. R¹ can also be piperidinyl, piperazinyl, morpholinyl and thiomorpholinyl. Additionally it can be pyrrolidinyl or tetrazolyl.

When Ar² represents an optionally substituted bicyclic aromatic or heteroaromatic it may be selected from isoquinolinyl, quinoxalinyl, benzoxazolyl, quinolinyl, napththyridinyl, benzofuranyl, benzimidazolyl, benzothienyl, indolyl, benzothiazoyl, quinazolinyl or benzoxazolyl. Additionally it can be naphthyl, benzotriazolyl or benzothiadiazolyl.

Preferably Ar² represents optionally substituted phenyl, pyridyl, thiazolyl, pyrazolyl, naphthyl or 1,2,3-triazolyl. Additionally Ar² represents thienyl and benzoxazolyl.

Even more preferably R¹ represents a trifluoromethoxy group, methoxy group, iodo, or an optionally substituted phenyl, pyridyl, pyrazolyl or oxadiazolyl group.

When X is CO, m is preferably 1 and p is preferably 0.

Optional substituents for the groups Ar¹, Ar² and R¹ include halogen, hydroxy, oxo, cyano, nitro, (C₁₋₄)alkyl, (C₁₋₄)alkoxy, halo(C₁₋₄)alkyl, halo(C₁₋₄)alkoxy, aryl(C₁₋₄)alkoxy, (C₁₋₄)alkylthio, hydroxy(C₁₋₄)alkyl, (C₁₋₄)alkoxy(C₁₋₄)alkyl, (C₃₋₆)cycloalkyl(C₁₋₄)alkoxy, (C₁₋₄)alkanoyl, (C₁₋₄)alkoxycarbonyl, (C₁₋₄)alkylsulfonyl, (C₁₋₄)alkylsulfonyloxy, (C₁₋₄)alkylsulfonyl(C₁₋₄)alkyl, arylsulfonyl, arylsulfonyloxy, arylsulfonyl(C₁₋₄)alkyl, (C₁₋₄)alkylsulfonamido, (C₁₋₄)alkylamido, (C₁₋₄)alkylsulfonamido(C₁₋₄)alkyl, (C₁₋₄)alkylamido(C₁₋₄)alkyl, arylsulfonamido, arylcarboxamido, arylsulfonamido(C₁₋₄)alkyl, arylcarboxamido(C₁₋₄)alkyl, aroyl, aroyl(C₁₋₄)alkyl, or aryl(C₁₋₄)alkanoyl group; a group R³R⁴N—, R³OCO(CH₂), R³CON(R⁴)(CH₂)ᵣ, R³R⁴NCO(CH₂)ᵣ, R³R⁴NSO₂(CH₂)ᵣ or R³SO₂NR⁴(CH₂)ᵣ where each of R³ and R⁴ independently represents a hydrogen atom or a (C₁₋₄)alkyl group or where appropriate R³R⁴ forms part of a (C₃₋₆)azacyloalkane or (C₃₋₆) (2-oxo)azacycloalkane ring and r represents zero or an integer from 1 to 4. Additional substituents are (C₁₋₄)acyl, aryl, aryl(C₁₋₄)alkyl, (C₁₋₄)alkylamino(C₁₋₄)alkyl, R³R⁴N(CH₂)n-, R³R⁴N(CH₂)nO—, wherein n represents an integer from 1 to 4. Additionally when the substituent is R³R⁴N(CH₂)n- or R³R⁴N(CH₂)nO, R³ with at least one CH₂ of the (CH₂)n portion of the group form a (C₃₋₆)azacycloalkane and R⁴ represents hydrogen, a (C₁₋₄)alkyl group or with the nitrogen to which it is attached forms a second (C₃₋₆)azacycloalkane fused to the first (C₃₋₆)azacycloalkane.

Preferred optional substituents for Ar² are halogen, cyano, (C₁₋₄)alkyl, (C₁₋₄)alkanoyl. Additional preferred optional substituents are hydroxy(C₁₋₄)alkyl, R³R⁴N(CH₂)n-, R³R⁴N—, or R³R⁴N(CH₂)nO—.

Preferred optional substituents for Ar¹ are halogen, cyano, (C₁₋₄)alkyl, hydroxy(C₁₋₄)alkyl, (C₁₋₄)acyl, (C₁₋₄)alkoxy(C₁₋₄)alkyl, R³R⁴NCO(CH₂)r-, R³R⁴N(CH₂)n-, R³R⁴N(CH₂)nO— or R³R⁴N—.

Preferred optional substituents for R¹ are halogen, cyano, R³R⁴N—, R³R⁴N(CH₂)n- and R³R⁴N(CH₂)nO—. Additional optional substituents are (C₁₋₄)alkyl, and (C₁₋₄)alkylamido.

In addition Ar¹ may be optionally substituted by a phenyl ring optionally substituted by a halogen, cyano, C₁₋₄alkanoyl or C₁₋₄alkylsulfonyl group; or by a 5- or 6-membered heterocyclic ring, optionally substituted by a (C₁₋₂)alkyl or R³R⁴N— group; wherein R³ and R⁴ are as defined above.

In the groups Ar¹ and Ar², substituents positioned ortho to one another may be linked to form a fused ring.

Illustrative compounds of the invention are selected from:

2-(1-(2-Phenoxy)ethyl)-1-(2-phenyl)benzoylpiperidine;

2-(1-(2-(4-Chloro)phenoxy)ethyl)-1-(2-phenyl)benzoylpiperidine;

2-(1-(2-Phenoxy)ethyl)-1-(2-triuoromethoxy)benzoylpiperidine;

2-(1-(2-Phenoxy)ethyl)-1-(2-(2-pyridyl))benzoylpiperidine;

2-(1-(2-Phenoxy)ethyl)-1-(2-(5-(3-methyl)-1,2,4-oxadiazolyl))benzoylpiperidine;

2-(1-(2-Phenoxy)ethyl)-1-(4-(2-methyl-5-phenyl)thiazolyl)carbonylpiperidine;

2-(1-(2-Phenoxy)ethyl)-1-(2-(4-fluoro)phenyl)benzoylpiperidine;

2-(1-(2-Phenoxy)ethyl)-1-(2-(2-cyano)phenyl)benzoylpiperidine;

2-(1-(2-Phenoxy)ethyl)-1-(2-(3-cyano)phenyl)benzoylpiperidine;

2-(1-(2-Phenoxy)ethyl)-1-(3-(2-phenyl)pyridyl)carbonylpiperidine;

2-(1-(2-(2-Cyano)phenoxy)ethyl)-1-(2-phenyl)benzoylpiperidine;

2-(1-(2-(3-Chloro)phenoxy)ethyl)-1-(2-phenyl)benzoylpiperidine;

2-(1-(2-(3,4-Dichloro)phenoxy)ethyl)-1-(2-phenyl)benzoylpiperidine;

2-(1-(2-(Phenoxy)ethyl)-1-(2-(1-pyrazolyl))benzoylpiperidine;

2-(1-(2-(4-Fluoro)phenoxy)ethyl)-1-(2-phenyl)benzoylpiperidine;

2-(1-(2-(4-Fluoro)phenoxy)ethyl)-1-(2-(2-pyridyl))benzoylpiperidine;

2-(1-(2-(4-Fluoro)phenoxy)ethyl)-1-(2-(5-(3-methyl)-1,2,4-oxadiazolyl))benzoylpiperidine;

2-(1-(2-(4-Fluoro)phenoxy)ethyl)-1-(4(2-methyl-5-phenyl)thiazolyl)carbonylpiperidine;

2-(1-(2-(3-Pyridyl)oxy)ethyl)-1-(4-(2-methyl-5-phenyl)thiazolyl)carbonylpiperidine;

2-(1-(2-(2-Pyridyl)oxy)ethyl)-1-(2-phenyl)benzoylpiperidine;

2-(1-(2-(2-Pyridyl)oxy)ethyl)-1-(2(2-pyridyl))benzoylpiperidine;

2-(1-(2-(2-Pyridyl)oxy)ethyl)-1-(2(5-(3-methyl)-1,2,4-oxadiazolyl))benzoylpiperidine;

2-(1-(2-(2-Pyridyl)oxy)ethyl)-1-(4-(2-methyl-5-phenyl)thiazolyl)carbonylpiperidine;

2-(1-(2-(1-Naphthyl)oxy)ethyl)1-(2-(2-pyridyl))benzoylpiperidine;

2-(1-(2-(1-Naphthyl)oxy)ethyl)-1-(2-(5-(3-methyl)-1,2,4-oxadiazolyl))benzoylpiperidine;

2-(1-(2-(1-Naphthyl)oxy)ethyl)-1-(4-(2-methyl-5-phenyl)thiazolyl)carbonylpiperidine;

2-(1-(2-(1-Naphthyl)oxy)ethyl)-1-(2-trifluoromethoxy)benzoylpiperidine;

2-(1-(2-(1-Naphthyl)oxy)ethyl)-1-(1-naphthoyl)piperidine;

2-(1-(2-(3-Cyano)phenoxy)ethyl)-1-(2-phenyl)benzoylpiperidine;

2-(1-(2-(3-Cyano)phenoxy)ethyl)-1-(4-(2-methyl-5-phenyl)thiazolyl)carbonylpiperidine;

E:Z-2-(1-(2-(3-Phenyl)propenyl)-1-(4-(2-methyl-5-phenyl)thiazolyl)carbonylpiperidine;

1-(4-(2-Methyl-5-phenyl)thiazolyl)carbonyl)-2-(1-(2-phenoxy)ethyl)-pyrrolidine;

1-(4-(2-Methyl-5-phenyl)thiazolyl)carbonyl)-2-(1-(2-phenoxy)ethyl)-1H-2,3,4,5,6,7-hexahydroazepine;

4-(4-(2-Methyl-5-phenyl)thiazolyl)carbonyl)-3-(1-(2-phenoxy)ethyl)-morpholine;

3-(1-(2-(4-Fluorophenoxy))ethyl)-4-(4-(2-methyl-5-phenyl)thiazolyl)carbonyl)morpholine;

2-(1-(2-Phenoxy)ethyl)-1-(2-phenyl)benzoylpyrrolidine;

2-(1-(2-Phenoxy)ethyl)-1-(2-phenyl)benzoyl-1H-2,3,4,5,6,7-hexahydroazepine;

2-(1-(2-Phenoxy)ethyl)-1-(2-(2-pyridyl))benzoyl-1H-2,3,4,5,6,7-hexahydroazepine;

2-(1-(2-Phenoxy)ethyl)-1-(2-(5-3-methyl)-1,2,4-oxadiazolyl))benzoyl-1H-2,3,4,5,6,7-hexahydroazepine;

2-(1-(2-Phenoxy)ethyl)-1-(2-trifluoromethoxy)benzoyl-1H-2,3,4,5,6,7-hexahydroazepine;

2-(1-(2-Phenoxy)ethyl)-1-(1-naphthoyl)-1H-2,3,4,5,6,7-hexahydroazepine;

3-(1-2-Phenoxy)ethyl)-4-(2-phenyl)benzoylmorpholine;

2-(1-(2-Phenoxy)ethyl)-1-(5-fluoro-2-(5-3-methyl)-1,2,4-oxadiazolyl))benzoylpiperidine;

1-(5-(3-Methyl-1-phenyl)-1H-pyrazolyl)carbonyl-2-(1-(2-phenoxy)ethyl)piperidine;

1-(4-(2-Methyl-5-phenyl)-2H-1,2,3-triazolyl)carbonyl-2-(1-(2-phenoxy)ethyl)piperidine;

1-(2-Iodo)benzoyl-2-(1-(2-phenoxy)ethyl)piperidine;

or a pharmaceutically acceptable salt of any one thereof.

Additional compounds of the invention are

(R,S)-1-Benzofuran-2-yl-2-(1-{1-[5-(4-fluoro-phenyl)-2-methyl-thiazol-4-yl]-methanol}-piperidin-2-yl)-ethanone;

1-Benzofuran-2-yl-2-(1-{1-[4-(4-fluoro-phenyl)-1-H-pyrazol-3-yl]-methanoyl}piperidin-2-yl)-ethanone;

1-Benzofuran-2-yl-2-(1-{1-[4-(4-fluoro-phenyl)-1-methyl-1H-pyrazol-3-yl]-methanoyl}-piperidin-2-yl)-ethanone;

1-Benzofuran-2-yl-2-{1-[1-(5-bromo-2-methoxy-phenyl)-methanoyl]-piperidin-2-yl}ethanone;

N-(3-{1-[2-(2-Benzofuran-2-yl-2-oxo-ethyl)-piperidin-1-yl]-methanoyl}-phenyl)-acetamide;

or a pharmaceutically acceptable salt of any one thereof.

Further compounds for the invention are;

1-(2-Benzyloxymethyl-piperidin-1-yl)-1-(2-methyl-5-phenyl-thiazol-4-yl)-methanone;

1-[2-(2-Benzyloxy-ethyl)-piperidin-1-yl]-1-(2-pyridin-2-yl-phenyl)-methanone;

1-[2-(2-Benzyloxy-ethyl)-piperidin-1-yl]-1-biphenyl-2-yl-methanone;

1-{2-[2-(4-Fluoro-benzyloxy)-ethyl]-piperidin-1-yl}-1-(2-methyl-5-phenyl-thiazol-4-yl)-methanone;

1-Biphenyl-2-yl-1-{2-[2-(4-fluoro-benzyloxy)ethyl]-piperidin-1-yl}methanone;

1-(2-Methyl-5-phenyl-thiazol-4-yl)-1-[2-(3-phenoxy-propyl)-piperidin-1-yl]-methanone;

1-[5-(4-Fluoro-phenyl)-2-methyl-thiazol-4-yl]-1-{2-[2-(pyridin-2-ylsulphanyl)-ethyl]-piperidine-1-yl}-methanone;

3-[1-(1-Biphenyl-2-yl-methanoyl)-piperidine-2-yl]-1-phenyl-propan-1-one;

1-[(S)-2-(5-Bromo-pyrimidin-2-yloxymethyl)-pyrrolidin-1-yl]-1-[2-(3-methyl-[1,2,4]oxadiazol-5-yl)-phenyl]-methanone;

or a pharmaceutically acceptable salt of any one thereof.

When a halogen atom is present in the compound of formula (I) it may be fluorine, chlorine, bromine or iodine.

When used herein the term aryl means a 5- to 6-membered ring, for example phenyl, or a 7- to 8-membered bicyclic ring system where at least one of the rings is aromatic, for example naphthyl.

When the compound of formula (I) contains an alkyl group, whether alone or forming part of a larger group, e.g. alkoxy or alkylthio, the alkyl group may be straight chain, branched or cyclic, or combinations thereof, it is preferably methyl or ethyl.

When X represents a group —CH═CH—, the compounds of formula (I) may exist as geometric isomers around the double bond. The present invention includes within its scope all such isomers, including mixtures.

It will be appreciated that compounds of formula (I) may exist as R or S enantiomers. The present invention includes within its scope all such isomers, including mixtures. Where additional chiral centres are present in compounds of formula (I), the present invention includes within its scope all possible diastereoismers, including mixtures thereof The different isomeric forms may be separated or resolved one from the other by conventional methods, or any given isomer may be obtained by conventional synthetic methods or by stereospecific or asymmetric syntheses.

It will be understood that the invention includes pharmaceutically acceptable derivatives of compounds of formula (I) and that these are included within the scope of the invention.

Particular compounds according to the invention include those mentioned in the examples and their pharmaceutically acceptable derivatives.

As used herein “pharmaceutically acceptable derivative” includes any pharmaceutically acceptable salt, ester or salt of such ester of a compound of formula (I) which, upon administration to the recipient is capable of providing (directly or indirectly) a compound of formula (I) or an active metabolite or residue thereof.

It will be appreciated that for use in medicine the salts of the compounds of formula (I) should be pharmaceutically acceptable. Suitable pharmaceutically acceptable salts will be apparent to those skilled in the art and include acid addition salts formed with inorganic acids e.g. hydrochloric, hydrobromic, sulphuric, nitric or phosphoric acid; and organic acids e.g. succinic, maleic, acetic, fumaric, citric, tartaric, benzoic, p-toluenesulfonic, methanesulfonic or naphthalenesulfonic acid. Other salts e.g. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further feature of the invention there is provided a process for the preparation of compounds of formula (I) and salts thereof. The following schemes detail some synthetic routes to compounds of the invention.

Scheme 1 is particularly applicable for compounds of formula (I) where X is O or S:

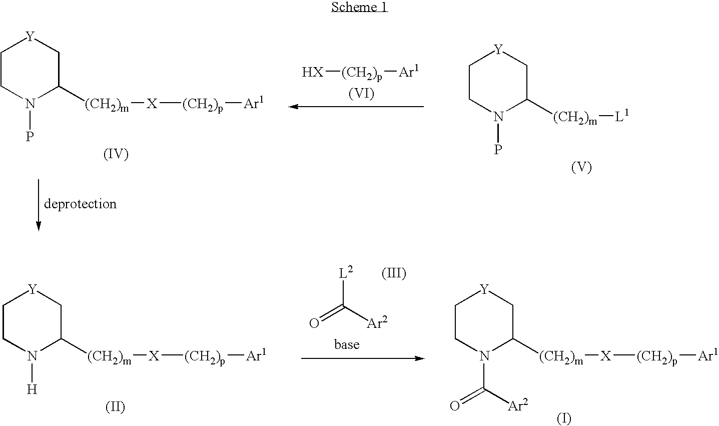

wherein Ar¹, Ar², Y, m, p and X are as defined for formula (I), L¹ and L² are leaving groups, and P is a protecting group.

Examples of suitable leaving groups L¹ include halogen, hydroxy, OSO₂Me, OSO₂(4-tolyl). The reaction of (V) with (VI) preferably proceeds in an inert solvent such as N,N-dimethylformamide in the presence of a base such as triethylamine, sodium hydride or potassium t-butoxide. In particular, when X is O and p is zero, L¹ is preferably hydroxy, and reaction of (V) with (VI) takes place under Mitsonobu conditions, i.e. in an inert solvent such as dichloromethane or tetrahydrofiran, in the presence of a phosphine reagent such as triphenylphosphine or tributylphosphine, and an azodicarbonyl reagent such as diethyl azodicarboxylate, diisopropyl azodicarboxylate, or 1,1′-azodicarbonyldipiperidine.

Examples of suitable leaving groups L² include halogen, hydroxy, OC(═O)alkyl and OC(═O)O-alkyl. The transformation (II) to (I) may be carried out in an inert solvent such as dichloromethane, in the presence of a base such as triethylamine. Alternatively this step may be carried out when L² represents hydroxy, in which case reaction with (II) takes place in an inert solvent such as dichloromethane in the presence of a diimide reagent such as 1-ethyl-3-(3-dimethylaminopropyl)carbodiimide hydrochloride, and an activator such as 1-hydroxybenzotriazole.

Examples of protecting groups P include t-butyloxycarbonyl, trifluoroacetyl and benzyloxycarbonyl. Deprotection conditions are respectively, acid (e.g. trifluoroacetic acid in dichloromethane), base (e.g. sodium hydroxide in a solvent such as aqueous methanol) and catalytic hydrogenolysis in an inert solvent (e.g using palladium on charcoal in a lower alcohol or ethyl acetate).

Compounds of formula (V) and (VI) are known in the literature or can be prepared by known methods.

Within the scheme above there is scope for functional group interconversion; for example in compound (V), conversion of one value of L¹ to another value of L¹; or conversion of one compound of formula (I) to another of formula (I) by interconversion of substituents (including interconversions of the residue X).

When R¹ is an aromatic group, the substituent R¹ may be introduced at the final stage as illustrated in Scheme 2 by reaction of a compound of formula (VII) where L³ represents a leaving group such as halogen (preferably bromo or iodo) or trifluoromethylsulfonyloxy, and all other variables are as previously defined, with a reagent R¹M, where M is the residue of an organometallic species e.g. B(OH)₂ or trialkylstannyl. Such a process may be carried out in an inert solvent such as 1,2-dimethoxyethane or 1,4-dioxan, in the presence of a transition metal catalyst such as Pd(PPh₃)₄.

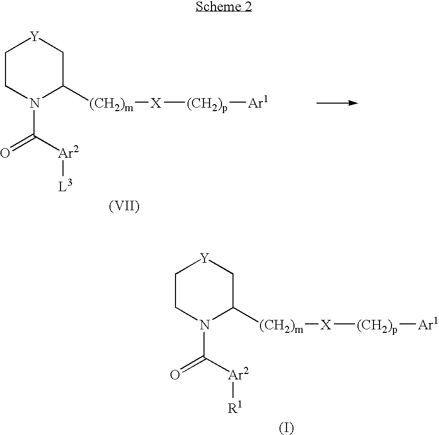

wherein Ar¹, Ar², Y, m, p and X are as defined for formula (I), L³ is a leaving group, and P is a protecting group.

Compounds of formula (I) where X represents —CH═CH— may be synthesised by the route shown in Scheme 3.

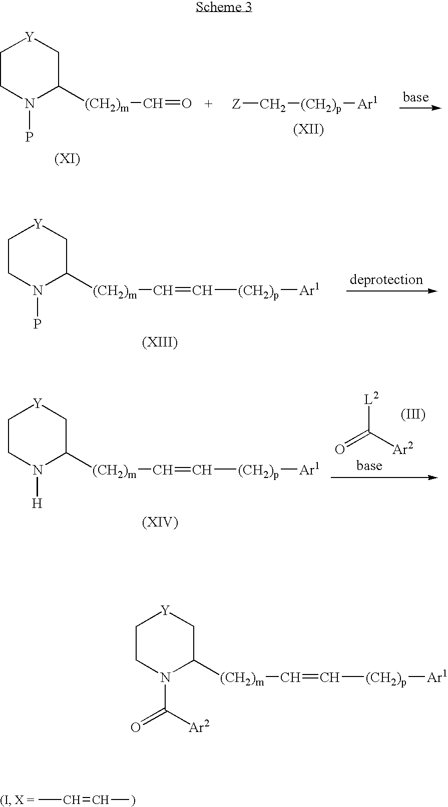

where Z represents a group [P⁺(aryl)₃][Br⁻] or a group P(═O)(Oalkyl)₂, and all other variables are as previously defined.

The reaction between (XI) and (XII) may be carried out in an inert solvent such as tetrahydrofuran, in the presence of a base such as butyllithium. Deprotection and final coupling steps can be carried out in a manner similar to those described in Scheme 1.

Compounds of formula (XI) and (XII) are known in the literature or can be prepared by known methods.

Compounds of formula (I) where X is C═O may be prepared by: reaction of a compound of formula (XI) with a compound T-(CH₂)ₚ—Ar¹, where T is the residue of an organometallic species, e.g. Li— or BrMg—, in an inert solvent such as tetrahydrofaran; followed by oxidation of the resulting secondary alcohol with an oxidant such as Dess Martin periodinane in an inert solvent such as dichloromethane; then deprotection and coupling of the resultant secondary amine with a compound of formula (E) in the manner previously described.

Alternatively compounds of formula (I) where X is C═O may be prepared by reaction of a compound of formula (XV);

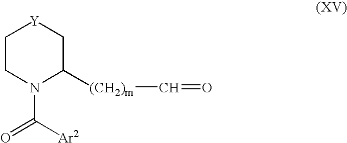

wherein Y, m and Ar² are as defined for formula (I); with a compound T-(CH₂)ₚ—Ar¹ as defined above in an inert solvent such as tetrahydrofuran; followed by oxidation of the resulting secondary alcohol with an oxidant such as Dess Martin periodinane as described above. Compounds of formula (XV) are known in the literature or can be prepared by known methods.

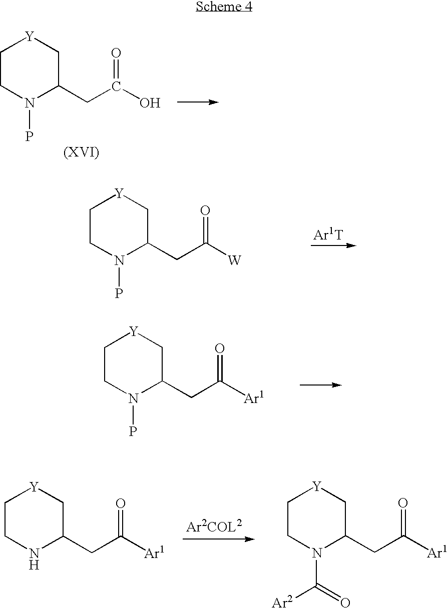

wherein Y, Ar¹ and Ar² are as defined for formula (I), and P represents a protecting group and L² is a leaving group as described for scheme 1.

W represents a leaving group as defined above or preferably a dialkylamino or N-methoxy-N-methyl group, and T is the residue of an organometallic species or metal such as lithium.

Compounds of structure (XVI) are known in the literature or are synthesised by known methods.

The compounds of formula (I) may be prepared singly or as compound libraries comprising at least 2, e.g. 5 to 1000, preferably 10 to 100 compounds of formula (I). Compound libraries may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable salts thereof

Pharmaceutically acceptable salts may be prepared conventionally by reaction with the appropriate acid or acid derivative.

The compounds of formula (I) and their pharmaceutically acceptable salts are useful for the treatment of diseases or disorders where an antagonist of a human orexin receptor is required such as obesity and diabetes; prolactinoma; hypoprolactinemia; hypothalamic disorders of growth hormone deficiency; idiopathic growth hormone deficiency; Cushing's syndrome/disease; hypothalamic-adrenal dysfunction; dwarfism; sleep disorders; sleep apnea; narcolepsy; insomnia; parasomnia; jet-lag syndrome; sleep disturbances associated with diseases such as neurological disorders, neuropathic pain and restless leg syndrome; heart and lung diseases; depression; anxiety; addictions; obsessive compulsive disorder; affective neurosis/disorder, depressive neurosis/disorder; anxiety neurosis; dysthymic disorder; behaviour disorder; mood disorder; sexual dysfunction; psychosexual dysfunction; sex disorder; sexual disorder, schizophrenia; manic depression; delerium; dementia; bulimia and hypopituitarism.

The compounds of formula (I) or pharmaceutically acceptable derivatives thereof are also useful in the treatment of stroke, particularly ischaemic or haemorrhagic stroke. Furthermore the compounds of formula (I) or pharmaceutically acceptable derivatives thereof are also useful in blocking the emetic response.

The compounds of formula (I) and their pharmaceutically acceptable derivatives are particularly useful for the treatment of obesity, including obesity associated with Type 2 diabetes, sleep disorders, stroke and blocking the emetic response for example nausea and vomiting.

Other diseases or disorders which may be treated in accordance with the invention include disturbed biological and circadian rhythms; adrenohypophysis disease; hypophysis disease; hypophysis tumor/adenoma; adrenohypophysis hypofunction; functional or psychogenic amenorrhea; adrenohypophysis hyperfunction; migraine; hyperalgesia; pain; enhanced or exaggerated sensitivity to pain such as hyperalgesia, causalgia and allodynia; acute pain; burn pain; atypical facial pain; neuropathic pain; back pain; complex regional pain syndromes I and E; arthritic pain; sports injury pain; pain related to infection e.g. HIV, post-polio syndrome and post-herpetic neuralgia; phantom limb pain; labour pain; cancer pain; post-chemotherapy pain; post-stroke pain; post-operative pain; neuralgia; and tolerance to narcotics or withdrawal from narcotics.

The invention also provides a method of treating or preventing diseases or disorders where an antagonist of a human orexin receptor is required, which comprises administering to a subject in need thereof an effective amount of a compound of formula (I), or a pharmaceutically acceptable derivative thereof.

The invention also provides a compound of formula (I), or a pharmaceutically acceptable derivative thereof, for use in the treatment or prophylaxis of diseases or disorders where an antagonist of a human orexin receptor is required.

The invention also provides the use of a compound of formula (I), or a pharmaceutically acceptable derivative thereof, in the manufacture of a medicament for the treatment or prophylaxis of diseases or disorders where an antagonist of a human orexin receptor is required.

For use in therapy the compounds of the invention are usually administered as a pharmaceutical composition. The invention also provides a pharmaceutical composition comprising a compound of formula (I), or a pharmaceutically acceptable derivative thereof, and a pharmaceutically acceptable carrier.

The compounds of formula (I) and their pharmaceutically acceptable derivative may be administered by any convenient method, e.g. by oral, parenteral, buccal, sublingual, nasal, rectal or transdermal administration, and the pharmaceutical compositions adapted accordingly.

The compounds of formula (I) and their pharmaceutically acceptable derivative which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochloro-hydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the compound of formula (I), or a pharmaceutically acceptable salt thereof, used in the treatment or prophylaxis of the abovementioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg; and such therapy may extend for a number of weeks or months. In the case of pharmaceutically acceptable salts the above figures are calculated as the parent compound of formula (I).

No toxicological effects are indicated/expected when a compound of formula (I) is administered in the above mentioned dosage range.

Human orexin-A has the amino acid sequence:

```
pyroGlu Pro Leu Pro Asp Cys Cys Arg Gln Lys Thr Cys Ser Cys Arg Leu
  1 5 10 15
Tyr Glu Leu Leu His Gly Ala Gly Asn His Ala Ala Gly Ile Leu Thr
                        20 25 30
Leu-NH₂
```

Orexin-A can be employed in screening procedures for compounds which inhibit the ligand's activation of the orexin-1 receptor.

In general, such screening procedures involve providing appropriate cells which express the orexin-1 receptor on their surface. Such cells include cells from mammals, yeast, Drosophila or E. coli. In particular, a polynucleotide encoding the orexin-1 receptor is used to transfect cells to express the receptor. The expressed receptor is then contacted with a test compound and an orexin-1 receptor ligand to observe inhibition of a functional response. One such screening procedure involves the use of melanophores which are transfected to express the orexin-1 receptor, as described in WO 92/01810.

Another screening procedure involves introducing RNA encoding the orexin-1 receptor into Xenopus oocytes to transiently express the receptor. The receptor oocytes are then contacted with a receptor ligand and a test compound, followed by detection of inhibition of a signal in the case of screening for compounds which are thought to inhibit activation of the receptor by the ligand.

Another method involves screening for compounds which inhibit activation of the receptor by determining inhibition of binding of a labelled orexin-1 receptor ligand to cells which have the receptor on their surface. This method involves transfecting a eukaryotic cell with DNA encoding the orexin-1 receptor such that the cell expresses the receptor on its surface and contacting the cell or cell membrane preparation with a compound in the presence of a labelled form of an orexin-1 receptor ligand. The ligand may contain a radioactive label. The amount of labelled ligand bound to the receptors is measured, e.g. by measuring radioactivity.

Yet another screening technique involves the use of FLIPR equipment for high throughput screening of test compounds that inhibit mobilisation of intracellular calcium ions, or other ions, by affecting the interaction of an orexin-1 receptor ligand with the orexin-1 receptor.

All publications, including but not limited to patents and patent applications, cited in this specification are herein incorporated by reference as if each individual publication were specifically and individually indicated to be incorporated by reference herein as though fully set forth.

The following Examples illustrate the preparation of pharmacologically active compounds of the invention. The Descriptions D1-D23 illustrate the preparation of intermediates to compounds of the invention.

In the Examples ¹H NMRs were measured at 250 MHz in CDCl₃ unless otherwise stated.

Abbreviations used herein are

MDC represents methylene dichloride

THF represents tetrahydrofuran

HATU represents O-(7-azabenzotriazol-1-yl)-N,N,N,N′,N′-tetramethyluronium hexafluorophosphate

DMF represents dimethyl fomiamide

Description 1(a): 1-(t-Butyloxycarbonyl)-2-(1-(2-(3,4-dichloro)phenoxy)-ethyl)piperidine

A mixture of 1-tert-butoxycarbonyl-2-piperidineethanol (1.50 g, 6.55 mmol), triphenylphosphine (1.72 g, 6.56 mmol), and 3,4-dichlorophenol (1.07 g, 6.56 mmol) in dry MDC (25 ml), was cooled in an ice-bath. Diethyl azodicarboxylate (1.03 ml, 1.14 g, 6.54 mmol) in dry MDC (7 ml), was added dropwise, with stirring under an argon atmosphere over 0.5 h. The reaction mixture was stirred at room temperature for 18 hour, then concentrated in vacuo. The residue was purified by column chromatography using silica gel (100 g) eluting with MDC. Fractions containing desired material were combined and concentrated in vacuo. The residue was dissolved in 1:1 ether-hexane (40 ml), and washed with 1 M aqueous sodium hydroxide. The organic layer was removed, dried (Na₂SO₄), filtered and the solvent removed in vacuo to give the title compound as a yellow oil (1.31 g, 53%). ¹H NMR δ: 1.39 (9H, s), 1.62 (6H, m), 1.83 (1H, m) 2.21 (1H, m), 2.79 (1H, m), 3.91 (2H, m), 4.01 (1H, m), 4.47 (1H, m) 6.72 (1H, dd, J=9 Hz, 3 Hz), 6.96 (1H, d, J=3 Hz), 7.30 (1H, d, J=9 Hz).

The following compounds were prepared in a similar manner to Description 1(a):

1(b): 1-(t-Butyloxycarbonyl)-2-(1-(2-phenoxy)ethyl)piperidine

¹H NMR δ: 1.40 (9H, s), 1.63 (6H, m), 1.87 (1H, m), 2.22 (1H, m), 2.82 (1H, m), 3.95 (2H, m), 4.05 (1H, m), 4.47 (1H, m), 6.81-6.96 (3H, m), 7.20-7.31 (2H, m).

1(c): 1-(t-Butyloxycarbonyl)-2-(1-(2-(1-naphthyl)oxy)ethyl)piperidine

¹H NMR δ: 1.34 (9H, s), 1.57-1.75 (6H, m), 2.05 (1H, m), 2.37 (1H, m), 2.89 (1H, m), 3.96-4.21 (3H, m), 4.60 (1H, m), 6.76 (1H, dd, J=7, 1 Hz), 7.26-7.53 (4H, m), 7.78 (1H, m), 8.27 (1H, m).

1(d): 1-(t-Butyloxycarbonyl)-2-1-(2-(3-cyano)phenoxy)ethyl)piperidine

¹H NMR δ: 1.38 (9H, s), 1.62 (6H, m), 1.86 (1H, m), 2.25 (1H, m) 2.81 (1H, m), 3.99 (3H, m), 4.51 (1H, m), 7.10 (2H, m), 7.23 (1H, m), 7.36 (1H, m).

1(e): 1-(t-Butyloxycarbonyl)-2-(1-(2-(2-pyridyl)oxy)ethyl)piperidine

¹H NMR δ: 1.39 (9H, s), 1.59 (6H, m), 1.88 (1H, m), 2.19 (1H, m), 2.80 (1H, m), 4.02 (1H, m), 4.29 (2H, m), 4.47 (1H, m), 6.72 (1H, m), 6.84 (1H, m), 7.55 (1H, m), 8.14 (1H, m).

1(f): 1-(t-Butyloxycarbonyl)-2-(1-(2-(4-fluoro)phenoxy)ethyl)piperidine

¹H NMR δ: 1.39 (9H, s), 1.62 (6H, m), 1.83 (1H, m), 2.22 (1H, m), 2.81 (1H, m), 3.89 (2H, m), 4.02 (1H, m), 4.47 (1H, m), 6.80 (2H, m) 6.95 (2H, m).

Description 2(a): 2-(1-(2-(3,4-Dichloro)phenoxy)ethyl)piperidine

A mixture of 1-(t-butyloxycarbonyl)2-(1-(2-(3,4-dichloro)phenoxy)ethyl)piperidine (1.31 g, 3.49 mmol), and trifluoroacetic acid (3.5 ml) in MDC (18 ml) was stirred at room temperature for 1 h. The reaction mixture was evaporated to dryness in vacuo, and the residue partitioned between 1:1 ether-hexane (30 ml) and 1 N HCl (30 ml). The aqueous layer was seperated and basified with 5 N NaOH to pH 14, and extracted with MDC (2×20 ml). The combined organic washes were dried (Na₂SO₄), filtered and evaporated in vacuo to give a yellow oil (0.80 g, 83%).

Mass Spectrum (API⁺): Found 274 (MH⁺). C₁₃H₁₇³⁵Cl₂NO requires 273. ¹H NMR δ: 1.17(1H, m), 1.39 (2H, m), 1.63 (3H, m), 1.82 (3H, m), 2.68 (2H, m), 3.07 (1H, m), 4.02 (2H, m), 6.75 (1H, dd, J=9 Hz, 3 Hz), 7.00 (1H, d, J=3 Hz), 7.30 (1H, d, J=9 Hz).

The following compounds were prepared in a similar manner to Description 2(a):

2(b): 2-(1-(2-Phenoxy)ethyl)piperidine

Mass spectrum (API⁺): Found MH⁺ 206. C₁₃H₁₉NO requires 205.

2(c): 2-(1-(2-(1-naphthyl)oxy)ethyl)piperidine

Mass spectrum (API⁺): Found MH⁺ 256. C₁₇H₂₁NO requires 255.

2(d): 2-(1-(2-(3-Pyridyl)oxy)ethyl)piperidine

Mass Spectrum (API⁺): Found 207. C₁₂H₁₈N₂O requires 206. ¹H NMR δ: 1.16 (1H, m), 1.38 (2H, m), 1.63 (3H, m), 1.83 (3H, m), 2.69 (2H, m), 3.07 (1H, m), 4.10 (2H, m), 7.19 (2H, m) 8.18 (1H, m), 8.31 (1H, m).

2(e): 2-(1-(2-(3-Cyano)phenoxy)ethyl)piperidine

Mass Spectrum (API⁺): Found 231. C₁₄H₁₈N₂O requires 230.

2(f): 2-(1-(2-(2-pyridy)oxy)ethyl)piperidine

Mass Spectrum (API⁺): Found 207. C₁₂H₁₈N₂O requires 206. ¹H NMR δ: 1.19-1.52 (4H, m), 1.64 (3H, m), 1.82 (2H, m), 2.65 (2H, m), 3.08 (1H, m), 4.36 (2H, m), 6.72 (1H, m), 6.83 (1H, m), 7.55 (1H, m), 8.12 (1H, m).

2(g): 2-(1-(2-(4-Fluoro)phenoxy)ethyl)piperidine

Mass Spectrum (API⁺): Found 224. C₁₃H₁₈ FNO requires 223. ¹H NMR δ: 1.18(1H, m), 1.39 (2H, m), 1.62 (3H, m), 1.81 (3H, m), 2.68 (2H, m), 3.07 (1H, m), 4.01 (2H, t, J=6 Hz), 6.83 (2H, m), 6.94 (2H, m).

2(h): 2-(1-(2-(3-Phenyl)propenyl))piperidine (2:1 Mixture of E:Z Isomers).

Mass spectrum (API⁺): Found MH⁺ 202. C₁₄H₁₉N requires 201.

Description 3: 1-(t-Butyloxycarbonyl)-2-(1-(2-(3-pyridy)oxy)ethyl)piperidine

A mixture of 1-tert-butoxycarbonyl-2-piperidineethanol (3.0 g, 13.2 mmol), triphenylphosphine (3.45 g, 13.2 mmol), and 3-hydroxypyridine (1.25 g, 13.2 mmol), in dry DMF (55 ml), was cooled to 0° C. in an ice-methanol bath. Diethyl azodicarboxylate (2.1 ml, 2.30 g, 13.2 mmol) was added and the reaction mixture stirred at room temperature under an argon atmosphere for 4 h. Most of the DMF was removed in vacuo and and the residue dissolved in MDC (100 ml), and washed with water (3×100 ml). The organic phase was separated, dried (Na₂SO₄), filtered and evaporated in vacuo to give a green oil (8.1 g) which was purified by chromatography on silica gel (˜200 g) eluting from 0-2% 0.880 ammonia in MDC. Fractions containing desired material were combined and evaporated in vacuo to give a brown oil (6.77 g). This material was further purified by passing through a prepacked SCX column eluting from 0-2% 0.880 ammonia in methanol. Fractions containing desired material were combined and evaporated in vacuo to give the title compound as a brown oil (1.23 g, 30%). Mass Spectrum (API⁺): Found 307. C₁₇H₂₆N₂O₃ requires 306. ¹H NMR δ: 1.38 (9H, s), 1.62 (6H, m), 1.85 (1H, m), 2.27 (1H, m), 2.81 (1H, m), 3.99 (3H, m), 4.51 (1H, m), 7.17 (2H, m), 8.20 (1H, m), 8.29 (1H, m).

Description 4: 1-(t-Butyloxycarbonyl)-2-(1-(2-(3-phenyl)propenyl))piperidine (2:1 Mixture of E:Z Isomers)

To a stirred suspension of benzyltriphenylphosphonium bromide (10.3 g, 23.8 mmol) in dry tetrahydrofuran (60 ml) at 20° C. under argon was added a solution of n-butyllithium in hexane (1.6 M, 11 ml, 17.3 mmol), dropwise over 0.5 hours. The resulting mixture was stirred at 20° C. for 0.5 hours, then a solution of 2-(2-(1-(t-butyloxycarbonyl)piperidinyl)acetaldehyde (2.61 g, 11.5 mmol) in dry tetrahydrofuran (40 ml) was added dropwise over 0.1 hours. The resulting mixture was stirred at 20° C. for 18 h, then poured into a mixture of water (100 ml) and brine (100 ml). The resulting suspension was extracted with dichloromethane (4×100 ml) and the combined organic extracts were dried (Na₂SO₄) and evaporated in vacuo to give a semi-solid. Chromatography on silica gel with dichloromethane elution gave the title compound (2.51 g, 73%) as a colourless oil. ¹H NMR δ: 1.30 and 1.47 (9H, 2 x s), 1.50-1.70 (6H, m), 2.31-2.90 (3H, m), 3.96 (1H, s), 4.36 (1H, s), 5.64 and 6.15 (1H, 2 x m), 6.41 and 6.50 (1, 2 x d, J=16 Hz and J=12 Hz, respectively), 7.13-7.38 (5H, m).

Description 5: (R,S)-2-[(Methoxy-methyl-carbamoyl)-methyl]-piperidine-1-carboxylic acid tert-butyl ester

A solution of 2-carboxymethyl-piperidine-1-carboxylic acid tert-butyl ester (2.29 g, 10 mmol) in DMF (20 ml) was treated sequentially with N,N-diisopropylethylamine (4.0 ml), HATU (3.8 g, 10 mmol) and O,N-dimethyl-hydroxylamine.HCl (0.98 g, 10 mmol). The reaction mixture was stirred under argon at room temperature for 16 h. The volatiles were removed in vacuo and the residue was chromatographed (silica gel, diethyl ether) to afford the title compound as a white solid (2.60 g, 90%). Mass Spectrum (API⁺): Found 187 (MH⁺-^{t}BOC). C₁₄H₂₆N₂O₄ requires 286.

Description 6: (R,S)-2-(2-Benzofuran-2-yl-2-oxo-ethyl)-piperidine-1-carboxylic acid tert-butyl ester

To a solution of benzofuran (0.95 g, 8.0 mmol) in THF (40 ml), under argon at −40° C. was added n-butyllithium (2.5M in hexanes) (4.00 ml, 10.0 mmol) over 5 min. The resultant mixture was stirred for 15 min. at −40° C., then (R,S)-2-[(methoxy-methyl-carbamoyl)-methyl]-piperidine-1-carboxylic acid tert-butyl ester, (2.30 g, 8.0 mmol) in THF (10 ml) was added over 1 min. and the resultant solution stirred for 20 min. at −40° C. The mixture was poured into saturated ammonium chloride (20 ml) and extracted with ethyl acetate (3×). The combined organics were dried (MgSO₄) and the solvent removed in vacuo. The resultant residue was chromatographed (silica gel, MDC) to afford the title compound (2.2 g, 84%). ¹H NMR δ: 1.35 (9H, s), 1.44 (1H, m), 1.65 (5H, m), 2.94 (1H, dt, J=3 and 13 Hz), 3.17 (2H, m), 4.05 (1H, broad d), 4.89 (1H, m), 7.31 (1H, t, J=8 Hz), 7.48 (1H, m), 7.57 (2H, m), 7.72 (1H, d, J=8 Hz).

Description 7: (R,S)1-Benzofuran-2-yl-2-piperidin-2-yl-ethanone

A stirring solution of (R,S)-2-(2-benzofuran-2-yl-2-oxo-ethyl)-piperidine-1-carboxylic acid tert-butyl ester, (1.68 g, 4.9 mmol) in MDC (20 ml) was treated with trifluoroacetic acid (5 ml). The mixture was stirred at 50° C. for 1 h, cooled and the volatiles removed in vacuo. The residue was dissolved in MDC and washed with saturated sodium bicarbonate, dried (MgSO₄) and the solvent removed in vacuo to afford the title compound (1.20 g, 99%). Mass Spectrum (API⁺): Found 244 (MH⁺). C₁₅H₁₉NO₂ requires 243.

Description 8: 2-(Benzyloxymethyl)-1-(t-butyloxycarbonyl)-piperidine

Sodium hydride (1.05 g, 26 mmol; 60% dispersion in oil) was added portionwise over 10 min. to a stirred mixture of 1-tert-butoxycarbonyl-2-piperidine methanol (5 g, 23 mmol) and benzyl bromide (2.8 ml, 24 mmol) in dry dimethylformamide (75 ml) at 0° C. under argon. After 3 h, the reaction mixture was poured into water (700 ml) and extracted with ethyl acetate (2×200 ml). Combined organics were washed with brine (2×200 ml), dried and evaporated. Chromatography on silica gel eluting with ethyl acetate-hexane mixtures afforded the title product as a solid (4.5 g, 63%). Mass Spectrum (API⁺): Found 206 (MH⁺-^{t}Boc). C₁₈H₂₇NO₃ requires 305.

Description 9: 2-(Benzyloxymethyl)-piperidine

A mixture of 2-(benzyloxymethyl)-1-(t-butyloxycarbonyl)-piperidine (4.5 g, 14.8 mmol) and trifluoroacetic acid (10 ml) in dry MDC (40 ml) was stirred at 35° C. for 1 h, cooled and evaporated. The residue was partitioned between MDC and 1M NaOH; the aqueous phase was extracted with MDC and the combined extracts dried and evaporated to afford the title product as a pale green oil (2.9 g, 96%). ¹H NMR δ: 1.00-1.80 (7H, m), 2.50-2.70 (1H, m), 2.70-2.85 (1H, m), 3.00-3.10 (1H, m), 3.27-3.36 (1H, m), 3.42-3.47 (1H, m), 4.51 (2H, s), 7.30-7.38 (5H, m).

Description 10(a): 2-(1-(2-(Benzyloxy)ethyl))-1-(t-butyloxycarbonyl)-piperidine

The title compound (0.318 g, 76%) was obtained from 1-t-butyloxycarbonyl-2-piperidineethanol (0.3 g, 1.32 mmol) and benzylbromide (0.17 ml, 1.3 mmol) according to the method of Description 8. Mass Spectrum (API⁺): Found 220 (MH⁺-^{t}Boc). C₁₉H₂₉NO₃ requires 319.

The following compounds were prepared in a similar manner to Description 10(a)

10(b): 2-(1-(2-(4-Fluorobenzyloxy)ethyl))-1-(t-butyloxycarbonyl)piperidine

Mass Spectrum (API⁺): Found 238 (MH⁺-^{t}-Boc). C₁₉H₂₈FNO₃ requires 337.

Description 11(a): 2-(1-(2-Benzyloxy)ethyl))piperidine

The title compound (0.22 g, 99%) was obtained from 2-(1-2-(benzyloxy)ethyl))-1-(t-butyloxycarbonyl)-piperidine (0.32 g, 1.0 mmol) according to the method of Description 9. Mass Spectrum (API⁺): Found 220 (MH⁺.) C₁₄H₂₁NO requires 219.

The following compounds were prepared in a similar manner to Description 11(a)

11(b): 2-(1-(2-(4-Fluorobenzyloxy)ethyl))piperidine

Mass Spectrum (API⁺): Found 238 (MH⁺). C₁₄H₂₀FNO requires 237.

Description 12: 2-(3-Hydroxypropan-1-yl)piperidine

2-Pyridinepropanol (15 g, 0.11 mol) in ethanol (200 ml) was hydrogenated at atmospheric pressure over platinum oxide catalyst (1.0 g) at 50° C. for 24 h. The reaction mixture was filtered through kieselguhr and the filtrate evaporated to afford the title product as an oil (16.7 g, 100%). Mass Spectrum (API⁺): Found 144. C₁₈H₁₇NO requires 143.

Description 13: 1-(t-Butyloxycarbonyol)-2-(3-hydroxypropan-1-yl)piperidine

To 2-3-hydroxypropan-1-yl)piperidine (16.7 g, 0.117 mol) in MDC (100 ml) was added di-tert-butyldicarbonate (25.5 g, 0.117 mol) followed by triethylamine (18 ml, 0.13 mol) at 0° C. The reaction mixture was allowed to reach ambient temperature and stirred for 16 h. The resulting mixture was poured onto silica gel and elution with MDC afforded the title product (8 g, 29%). Mass Spectrum (API⁺): Found 244 (Mt). C₁₃H₂₅NO₃ requires 243.

Description 14: 1-(t-Butyloxyearbonyl)-2-(3-phenoxy)propyl)piperidine

The title product (3.45 g, 51%) was obtained from 1-(t-butyloxycarbonyl)-2-3-hydroxypropan-1-yl)piperidine (5.15 g, 21.2 mmol) and phenol (2.0 g, 21.2 mmol) according to the method of Description 1. Mass Spectrum (API⁺): Found 220 (MH⁺-^{t}Boc). C₁₉H₂₉NO₃ requires 319.

Description 15: 2-(1-(3-Phenoxy)propyl)piperidine

The title compound (1.74 g, 74%) was obtained from 1-t-butyloxycarbonyl)-2-(1-(3-phenoxy)propyl)piperidine (3.42 g, 10.7 mmol) according to the method of Description 2(a). Mass Spectrum (API⁺): Found 220 (MH⁺). C₁₄H₂₁NO requires 219.

Description 16: 2-(2-Bromoethyl)-piperidine-1-carboxylic acid tert-butyl ester

To a mixture of 1-tert-butyloxycarbonyl-2-piperidine ethanol (6 g, 0.026 mol) and triphenylphosphine (11 g, 0.042 mol) in anhydrous tetrahydrofulm (100 ml) at 0° C. was added portionwise N-bromosuccinimide (7.5 g, 0.042 mol). The reaction mixture was allowed to reach ambient temperature, stirred for 64 h, and then evaporated to low volume prior to pouring onto silica gel and eluting with 30% ether-petrol (40-60°) to afford the title product (7.8 g, 99%) as an oil. ¹H NMR δ: 1.3-1.8 (6H, m), 1.47 (9H, s), 1.90-1.94 (1H, m), 2.31-2.35 (1H, m), 2.70-2.80 (1H, m), 3.31-3.38 (2H m), 3.95-4.05 (1H, m), 4.38-4.40 (1H, m).

Description 17: 2-[2-(Pyridin-2-ylsulphanyl)-ethyl]-piperidine-1-carboxylic acid tert-butyl ester

A mixture of 2-2-bromoethyl)-piperidine-1-carboxylic acid tert-butyl ester (1.7 g, 5.8 mmol), 2-mercaptopyridine (0.71 g, 6.4 mmol), lithium hydroxide (0.15 g, 6.4 mmol) and sodium iodide (1.92 g, 12.8 mmol) in dimethylformamide (17 ml) was stirred at ambient temperature for 18 h. The reaction mixture was diluted with ethyl acetate and washed with water (×3), brine, dried and evaporated. Chromatography on silica gel eluting with ethyl acetate-hexane mixtures afforded the title product (0.45 g, 27%). Mass Spectrum (Electrospray LC/MS) : Found 345 (MNa⁺). C₁₇H₂₆N₂O₂S requires 322.

Description 18: 2-(2-Piperidin-2-yl-ethylsulphanyl)-pyridine

The title compound (0.35 g, 99%) was obtained from 2-[2-(yridin-2-ylsulphanyl)-ethyl]-piperidine-1-carboxylic acid tert-butyl ester (0.44 g, 1.4 mmol) according to the method of Description 9. ¹H NMR δ: 1.05-1.20 (1H, m), 1.25-1.50 (2H, m), 1.50-1.60 (1H, m), 1.65-1.80 (5H, m), 2.40-2.60 (2H, m), 3.00-3.10 (1H, m), 3.15-3.25 (2H, m), 6.94-6.97 (1H, m), 7.14-7.18 (1H, m), 7.44-7.48 (1H, m), 8.40-8.41 (1H, m).

Description 19: 1-Biphenyl-2-yl-2-[2-(3-hydroxy-propyl)piperidin-1-yl]-methanone

A mixture of the amine HBr salt of description 12 (2.0 g, 8.9 mmol), biphenyl-2-carbonyl chloride (2.17 g, 10 mmol) and triethylamine (2.02 g, 20 mmol) in MDC (100 ml) was stirred at ambient temperature for 20 h, washed with saturated sodium hydrogen carbonate (200 ml) and the organics dried (Na₂SO₄) and evaporated in vacuo. The residue was chromatographed on silica using a 10-100% ethyl acetate in hexane gradient to afford the title compound as an oil. Mass Spectrum API⁺: Found 324 (MH⁺). C₂₁H₂₅NO₂ requires 323.

Description 20: 3[1-(1-Biphenyl-2-yl-methanoyl)-piperidine-2-yl]-propionaldehyde

To a solution of oxalyl chloride (1.4 g, 11 mmol) in MDC (40 ml) at −70° C. under argon was added a solution of dimethylsulfoxide (1.9 g, 24 mmol) in MDC (10 ml) dropwise over 0.2 h. The mixture was stirred at −70° C. for 1 h before a solution of the alcohol of description D19 (3.28 g, 10 mmol) in MDC (10 ml) was added dropwise over 0.1 h. The mixture was stirred at ζ70° C. for 1 h, then triethylamine (7.5 ml, 53.8 mmol) was added dropwise over 1 h. The resulting mixture was allowed to warm to ambient temperature and stirred for 1 h, then was evaporated in vacuo. The residue was partitioned between 1:1 ether-hexane (300 ml) and water (100 ml) and the organic phase washed with water (4×100 ml), dried (Na₂SO₄) and evaporated in vacuo. The residue was chromatographed on silica eluting with a 10-100% diethyl ether in hexane gradient to afford the title compound as a colourless oil (1.74 g). Mass Spectrum (API⁺): Found 322 (MH⁺). C₂₁H₂₃NO₂ requires 321.

Description 21: 1-Biphenyl-2-yl-1-[2-(3-hydroxy-3-phenyl-propyl)-piperidin-1-yl]-methanone

1.8M Phenyl lithium in cyclohexane/ether (1.2 ml, 2.16 mmol) was added dropwise over 0.25 h to a stirred solution of 3-[1-(1-biphenyl-2-yl-methanoyl)-piperidine-2-yl]-propionaldehyde (D20) (0.6 g, 1.87 mmol) in anhydrous ether (25 ml) at −70° C. under argon. After stirring at −70° C. for 1 h and at ambient temperature for 3 h, water (70 ml) was added and the mixture extracted with ethyl acetate (2×50 ml). Combined organics were dried (Na₂SO₄) and evaporated in vacuo to afford the title compound as a yellow gum (0.7 g, 94%). Mass spectrum (API⁺): Found 400 (MH⁺). C₂₇H₂₉NO₂ requires 399.

Description 22: (S)-2-(5-Bromo-pyrimidin-2-yloxymethyl)-pyrrolidine-1-carboxylic acid tert-butyl ester

A mixture of 2-chloro-5-bromo pyrimidine (1.48 g, 7.6 mmol), (S)-1-tert-butoxycarbonyl)-2-pyrrolidine methanol (2.0 g, 9.9 mmol), dibenzo-18-crown-6 (0.3 g, 0.83 mmol), diisopropylamine (3 ml, 17 mmol) and potassium hydroxide (0.73 g, 13 mmol) was heated in xylene (40 ml) at 130° C. for 3 days. The reaction mixture was then evaporated and partitioned between MDC and water. The organic phase was separated, washed with brine, dried (Na₂SO₄) and evaporated. Chromatography of the residue on silica gel eluting with ethyl actetate-pentane mixtures afforded the title product (0.573 g; 21%). Mass Spectrum (Electrospray LC/MS): Found 380 (MNa⁺). C₁₄H₂₀⁷⁹BrN₃O₃ requires 357.

Description 23: 5Bromo-2-((S)-1-pyrrolidin-2-ylmethoxy)-pyrimidine hydrochloride

A solution of (S-2-(5-bromo-pyrimidin-2-yloxymethyl)-pyrrolidine-1-carboxylic acid tert-butyl ester (0.57 g, 1.6 mmol) in methanol (12 ml) containing 4N HCl in dioxan (4 ml) was stirred at ambient temperature for 18 h., evaporated and dried in vacuo to afford the title product as a pale yellow solid (0.395 g, 96%). Mass Spectrum (Electrospray LC/MS): Found 258 (MH⁺). C₉H₁₂⁷⁹BrN₃O requires 257.

### EXAMPLE 1

A mixture of 2-(1-(2-phenoxy)ethyl)piperidine (0.10 g, 0.49 mmol), triethylamine (0.07 ml, 0.05 g, 0.50 mmol) and 2-biphenylcarbonyl chloride (0.10 g, 0.46 mmol) in dichloromethane (8 ml) was shaken at 20° C. for 1 h, then saturated aqueous NaHCO₃ was added and shaking continued for 0.1 hours. The organic phase was allowed to separate, then was applied directly to a pre-packed silica cartridge. Elution with 30-100% ethyl acetate-hexane gave 2-(12-phenoxy)ethyl)-1-(2-phenyl)benzoylpiperidine (0.137 g, 77%) as a colourless oil.

The compounds of the Examples below were prepared from the appropriate amine and acid chloride using a procedure similar to the following illustration for Example 1:

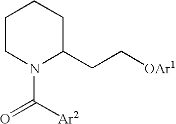

| Example | Ar² | Ar¹ | Mass Spectrum (API⁺). |
|---|---|---|---|
| 1 | 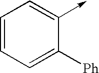 | -Ph | Found MH⁺386. C₂₆H₂₇NO₂ requires 385 |
| 2 | 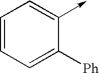 | 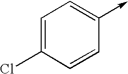 | Found MH⁺420. C₂₆H₂₆³⁵CINO₂ requires 419. |
| 3 | 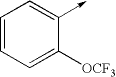 | -Ph | Found MH⁺394. C₂₁H₂₂F₃NO₃ requires 393. |
| 4 | 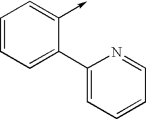 | -Ph | Found MH⁺387. C₂₅H₂₆N₂O₂ requires 386. |
| 5 | 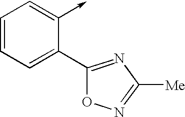 | -Ph | Found MH⁺392. C₂₃H₂₅N₃O₃ requires 391. |
| 6 | 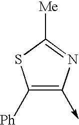 | -Ph | Found MH⁺407. C₂₄H₂₆N₂O₂S requires 406. |
| 7 | 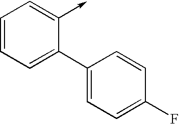 | -Ph | Found MH⁺404. C₂₆H₂₆FNO₂ requires 403. |
| 8 | 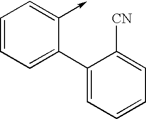 | -Ph | Found MH⁺411. C₂₇H_{26 N}₂O₂ requires 410. |
| 9 | 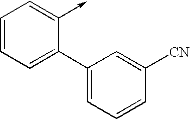 | -Ph | Found MH⁺411. C₂₇H₂₆N₂O₂ requires 410. |
| 10 | 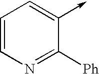 | -Ph | Found MH⁺387. C₂₅H₂₆N₂O₂ requires 386. |
| 11 | 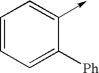 | 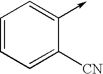 | Found MH⁺411. C₂₇H₂₆N₂O₂ requires 410. |
| 12 | 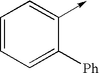 | 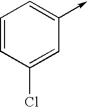 | Found MH⁺420. C₂₆H₂₆³⁵CINO₂ requires 419. |
| 13 | 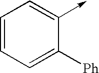 | 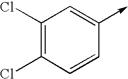 | Found MH⁺454. C₂₆H₂₅³⁵CI₂NO2 requires 453. |
| 14 | 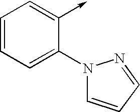 | -Ph | Found MH⁺376. C₂₆H₂₅N₃O₂ requires 375. |
| 15 | 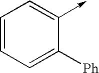 | 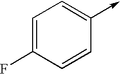 | Found MH⁺404. C₂₆H₂₆FNO₂ requires 403. |
| 16 | 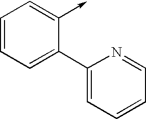 | 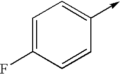 | Found MH⁺405. C₂₅H₂₅FN₂O₂ requires 404. |
| 17 | 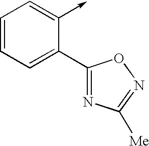 | 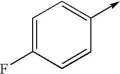 | Found MH⁺410. C₂₃H₂₄FN₃O₃ requires 409. |
| 18 | 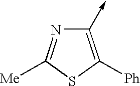 | 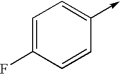 | Found MH⁺425. C₂₄H₂₅FN₂O₂S requires 424. |
| 19 | 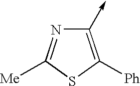 | 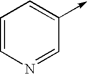 | Found MH⁺408. C₂₃H₂₅N₃O₂S requires 407. |
| 20 | 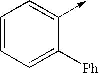 | 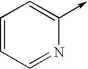 | Found MH⁺387. C₂₅H₂₆N₂O₂ requires 386. |
| 21 | 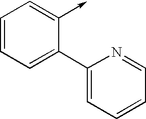 | 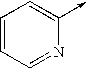 | Found MW 388. C₂₄H₂₅N₃O₂ requires 387. |
| 22 | 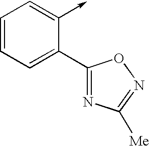 | 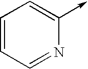 | Found MH⁺393. C₂₂H₂₄N₄O₃ requires 392. |
| 23 | 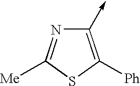 | 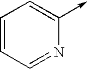 | Found MH⁺408. C₂₃H₂₅N₃O₂S requires 407. |
| 24 | 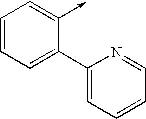 | 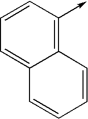 | Found MH⁺347. C₂₉H₂₈N₂O₂ requires 436. |
| 25 | 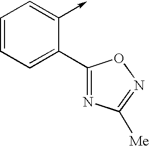 | 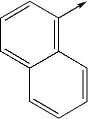 | Found MH⁺442. C₂₇H₂₁N₃O₃ requires 441. |
| 26 | 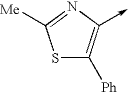 | 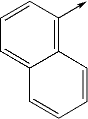 | Found MH⁺457. C₂₈H₂₈N₂O₂S requires 456 |
| 27 | 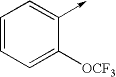 | 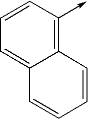 | Found MH⁺444. C₂₅H₂₄F₃NO₃ requires 443. |
| 28 | 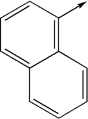 | 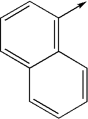 | Found MH⁺410. C₂₈H₂₇NO₂ requires 409. |
| 29 | 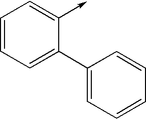 | 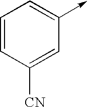 | Found MH⁺411. C₂₇H₂₆N₂O₂requires 410. |
| 30 | 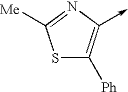 | 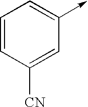 | Found MH⁺432. C₂₅H₂₅N₃O₂S requires 431. |

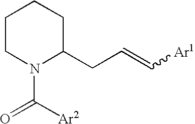

| | | | |
|---|---|---|---|
| 31 | 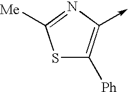 | -Ph | Found MH⁺403. C₂₅H₂₆N₂OS requires 402. |

### EXAMPLE 32

### 1-(4-(2-Methyl-5-phenyl)thiazolyl)carbonyl)-2-1-(2-phenoxy)ethyl)pyrrolidine

A mixture of 2-1(2-phenoxy)ethyl)pyrrolidine (0.029 g, 0.15 mmol), triethylamine (0.046 g, 0.45 mmol) and 4-(2-methyl-5-phenyl)thiazolyl)carbonyl chloride (0.043 g, 0.18 mmol) in dichloromethane (4 ml) was shaken at 20° C. for 1 hour, then saturated aqueous NaHCO₃ was added and shaking continued for 0.1 hour. The organic phase was allowed to separate, then was applied directly to a pre-packed silica cartridge. Elution with 10-100% ethyl acetate-hexane gave the title compound (0.034 g, 58%) as a tan oil. Mass Spectrum (Electrospray LC/MS): Found 393 (MH⁺). C₂₃H₂₄N₂O₂S requires 392. ¹H NMR δ: 1.5-2.1 (6H, m), 2.46 and 2.72 (3H, 2 x s), and 3.02 (1H, 2 x m), 3.21 and 3.62 (1H, 2 x m), 3.70 and 4.06 (2H, 2 x t, J=5 Hz), 3.90 and 4.40 (1H, 2 x m), 6.70-7.00 (3H, m), 7.2-7.6 (7H, m).

### EXAMPLE 33

### 1-(4-(2-Methyl-5-phenyl)thiazolyl)carbonyl)-2-(1-(2-phenoxy)ethyl)-1H-2,3,4,5,6,7-hexahydroazepine

A mixture of 2-(1-(2-phenoxy)ethyl)-1H-2,3,4,5,6,7-hexahydroazepine (0.033 g, 0.15 mmol), triethylamine (0.046 g, 0.45 mmol) and 4-(2-methyl-5-phenyl)thiazolyl)carbonyl chloride (0.043 g, 0.18 mmol) in dichloromethane (4 ml) was shaken at 20° C. for 1 hour. then saturated aqueous NaHCO₃ was added and shaking continued for 0.1 hour. The organic phase was allowed to separate, then was applied directly to a pre-packed silica cartridge. Elution with 10-100% ethyl acetate-hexane gave the title compound (0.055 g, 87%) as a tan oil. Mass Spectrum (Electrospray LC/MS): Found 421 (MH⁺). C₂₅H₂₈N₂O₂S requires 420. ¹H NMR δ: 1.10-2.20 (10H, m), 2.46 and 2.72 (3H, 2 x s), 2.65 and 2.90 (1H, 2 x m), 3.37 and 4.31 (1H, 2 x m), 3.6-3.9 (2H, m), 3.96 and 4.76 (1H, m), 6.70-7.00 (3H, m), 7.20-7.40 (5H, m), 7.50-7.60 (2H, m).

### EXAMPLE 34

### 4-(4-(2-Methyl-5-phenyl)thiazolyl)carbonyl)-3-(1-(2-phenoxy)ethyl)-morpholine

A mixture of 3-(1(2-phenoxy)ethyl)morpholine (0.027 g, 0.13 mmol), triethylamine (0.030 g, 0.30 mmol) and 4-(2-methyl-5-phenyl)thiazolyl)carbonyl chloride (0.033 g, 0.14 mmol) in dichloromethane (5 ml) was shaken at 20° C. for 1 hour, then saturated aqueous NaHCO₃ was added and shaking continued for 0.1 hour. The organic phase was allowed to separate, then was applied directly to a pre-packed silica cartridge. Elution with 10-100% ethyl acetate-hexane and then 2% methanol-ethyl acetate gave the title compound (0.048 g, 95%) as an oil. Mass Spectrum (Electrospray LC/MS): Found 409 (MH⁺). C₂₃H₂₄N₂O₃S requires 408.

### EXAMPLE 35

### 3(1-(2-(4-Fluorophenoxy))ethyl)-4-(4-(2-methyl-5-phenyl) thiazolyl)carbonyl)morpholine

A mixture of 3-(1-(2-(4-fluorophenoxy))ethyl)morpholine (0.027 g, 0.12 mmol), triethylamine (0.030 g, 0.30 mmol) and 4-(2-methyl-5-phenyl)thiazolyl)carbonyl chloride (0.029 g, 0.12 mmol) in dichloromethane (5 ml) was shaken at 20° C. for 1 hour, then saturated aqueous NaHCO₃ was added and shaking continued for 0.1 hour. The organic phase was allowed to separate, then was applied directly to a pre-packed silica cartridge. Elution with 10-100% ethyl acetate-hexane and then 2% methanol-ethyl acetate gave the title compound (0.039 g, 79%) as an oil. Mass Spectrum (Electrospray LC/MS): Found 427 (MH⁺). C₂₃H₂₃FN₂O₃S requires 426.

Compounds in Table 2 were prepared from the appropriate amine and acid chloride using a procedure similar to that described for Examples 31-35.

TABLE 2

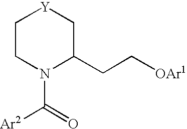

| | | | | Mass Spectrum |
|---|---|---|---|---|
| Example | Y | Ar² | Ar¹ | (Electrospray LC/MS) |
| 36 | bond | 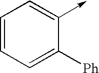 | Ph | Found MH⁺372. C₂₅H₂₅NO₂ requires 371 |
| 37 | (CH₂)₂ | 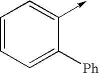 | Ph | Found MH⁺400. C₂₇H₂₉NO₂ requires 399 |
| 38 | (CH₂)₂ | 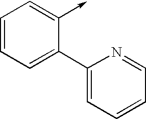 | Ph | Found MH⁺401. C₂₆H₂₈N₂O₂ requires 400 |
| 39 | (CH₂)₂ | 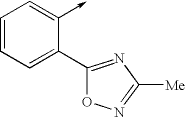 | Ph | Found MH⁺406. C₂₄H₂₇N₃O₃ requires 405 |
| 40 | (CH₂)₂ | 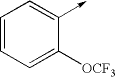 | Ph | Found MH⁺408. C₂₂H₂₄F₃NO₃ requires 407 |
| 41 | (CH₂)₂ | 1-naphthyl | Ph | Found MH⁺374. C₂₅H₂₇NO₂ requires 373 |
| 42 | O | 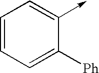 | Ph | Found MH⁺388. C₂₅H₂₅NO₃ requires 387 |
| 43 | CH₂ | 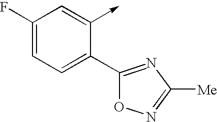 | Ph(4-F) | Found MH⁺428. C₂₃H₂₃F₂N₃O₃ requires 427 |
| 44 | CH₂ | 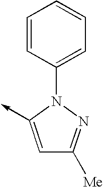 | -Ph | Found MH⁺390. C₂₄H₂₁N₃O₂ requires 389. |
| 45 | CH₂ | 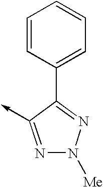 | -Ph | Found MH⁺391. C₂₃H₂₆N₄O₂ requires 390. |
| 46 | CH₂ | 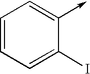 | -Ph | Found MH⁺436. C₂₀H₂₂¹²⁷INO₂ requires 435. |

### EXAMPLE 7

### (R,S)-1-Benzofuran-2-yl-2-(1-{1-[5-(4-fluoro-phenyl)-2-methyl-thiazol-4-yl]-methanoyl}-piperidin-2-yl)-ethanone

The title compound (0.120 g, 79%) was prepared from 5-(4-fluoro-phenyl)2-methyl-thiazole-4-carbocylic acid (0.079 g, 0.33 mmol) and (R,S)-1-benzofuran-2-yl-2-piperidin-2-yl-ethanone, (0.081 g, 33 mmol) according to a procedure similar to that for Description 5. Mass Spectrum (API⁺): found 463 (MH⁺). C₂₆H₂₃FN₂O₃S requires 462.

In a similar were prepared the compounds of Examples 48-51.

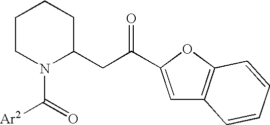

| Example | Ar² | Mass Spectrum (API⁺) |
|---|---|---|
| 48 | 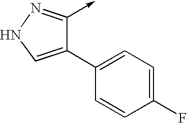 | Found 432 (MH⁺). C₂₅H₂₂FN₃O₃ requires 431 |
| 49 | 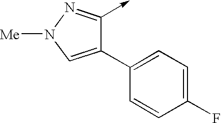 | Found 446 (MH⁺). C₂₆H₂₄FN₃O₃ requires 445 |
| 50 | 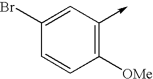 | Found 456 (MH⁺). C₂₃H₂₂Br⁷⁹NO₄ requires 455 |
| 51 | 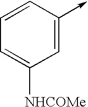 | Found 405 (MH⁺). C₂₄H₂₄N₂O₄ requires 404. |

### EXAMPLE 52

### 1-2-Benzyloxymethyl-piperidin-1-yl)-1-(2-methyl-5-phenyl-thiazol-4-yl)-methanone

The title compound (0.005 g, 13%) was obtained from 2-(benzyloxymethyl)-piperidine (0.020 g, 0.1 mmol) and 4-(2-methyl-5-phenyl)thiazolyl)carbonyl chloride (0.026 g, 0.12 mol) as described for Example 32. Mass Spectrum (Electrospray LC/MS): Found 407 (MH⁺). C₂₄H₂₆N₂O₂S requires 406.

### EXAMPLE 53

### 1-[2-(2-Benzyloxethyl)-piperidin-1-yl]-1-(2-pyridin-2-yl-phenyl)-methanone

The title compound (0.030 g, 67%) was obtained from 2-(1(2-benzyloxy)ethyl))piperidine (0.025 g, 0.11 mmol) and 2-(pyridin-2-yl) benzoyl chloride hydrochloride (0.030 g, 0.12 mmol) according to the method of Example 32. Mass Spectrum (Electrospray LC/MS): Found 401 (MH⁺). C₂₆H₂₈N₂O₂ requires 400.

The compounds of the Examples below were prepared from the appropriate amine and acid using similar procedures to those described above:

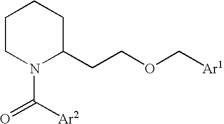

| | | | Mass Spectrum (Electrospray |
|---|---|---|---|
| Example | Ar² | Ar^{1 LC/MS), API}⁺ | |
| 54 | 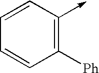 | 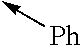 | Found 400 (MH⁺). C₂₇H₂₉NO₂requires 399. |
| 55 | 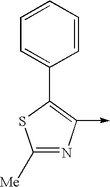 | 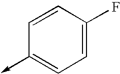 | Found 439 (MH⁺). C₂₅H₂₇FN₂O₂S requires 438. |
| 56 | 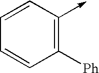 | 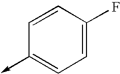 | Found 418 (MH⁺). C₂₇H₂₈FNO₂requires 417. |

### EXAMPLE 57

### 1-(2-Methyl-5-phenyl-thiazol-4-yl)-1-[2-(3-phenoxy-propyl)-piperidin-1-yl]-methanone

The title compound (0.021 g, 98%) was obtained from 2-(1-(3-phenoxy)propyl)piperidine (0.011 g, 0.05 mmol) and 4-(2-methyl-5-phenyl)thiazolyl)carbonyl chloride (0.013 g, 0.06 mmol) according to the method of Example 35. Mass Spectrum (API⁺): Found 421 (MH⁺). C₂₅H₂₈N₂O₂S requires 420.

### EXAMPLE 58

### 1-[5-(4-Fluoro-phenyl)-2-methyl-thiazol-4-yl]-1-{2-[2-(pyridin-2-ylsulphanyl)-ethyl]-piperidine-1-yl}-methanone

The title compound (0.091 g, 40%) was obtained from 2-(2-piperidin-2-yl-ethylsulphanyl)-pyridine (0.116 g, 0.5 mmol) and 5-(4-fluorophenyl)-2-methylthiazole-4-carbonyl chloride (0.146 g, 0.57 mmol) according to the method of Example 32. Mass Spectrum (Electrospray LC/MS): Found 442 (MH⁺). C₂₃H₂₄FN₃OS₂ requires 441.

### EXAMPLE 59

### 3-[1-(1-Biphenyl-2-yl-methanoyl)-piperidine-2-yl]-1-phenyl-propan-1-one

Dess-Martin periodinane (0.85 g, 2 mmol) was added over 0.16 h to a stirred solution of the alcohol of Description D21 (0.7 g, 1.75 mmol) in MDC (20 ml). After 1.16 h MDC was added and the mixture was washed with a 2:1 mixture of saturated sodium hydrogen carbonate: 10% sodium sulphite solution (2×100 ml). The organics were dried (Na₂SO₄), evaporated in vacuo and the residue chromatographed on silica eluting with a 0-30% ethyl acetate in hexane gradient to afford the title compound as a colourless gum (0.24 g, 34%). Mass Spectrum API⁺: Found 398 (MH⁺). C₂₇H₂₇NO₂ requires 397.

### EXAMPLE 60

### 1-[(S)-2-(5-Bromo-pyrimidin-2-yloxymethyl)-pyrrolidin-1-yl]-1-[2-(3methyl-[1,2,4]oxadiazol-5-yl)-phenyl]-methanone

To a solution of 5-bromo-2-(S)-1-pyrrolidin-2-ylmethoxy)-pyrimidine hydrochloride (0.09 g, 0.3 mmol) in dimethylformamide (1 ml) was added HATU (0.116 g, 0.3 mmol) and diisopropylethylamine (5 ml). After 18 h at ambient temperature, the reaction mixture was evaporated, and the residue chromatographed on silica gel, eluting with ethyl acetate-pentane mixtures, to afford the title compound as a gum (0.065 g, 48%). Mass Spectrum (Electrospray LC/MS): Found 444 (MH⁺), 466 (MNa⁺). C₁₉H₁₈⁷⁹BrN₅O₃ requires 443.

It is understood that the present invention covers al combinations of particular and preferred groups described herein above.

### Determination of Orexin-1 Receptor Antagonist Activity

The orexin-1 receptor antagonist activity of the compounds of formula (I) was determined in accordance with the following experimental method.

### Experimental Method

HEK293 cells expressing the human orexin-1 receptor were grown in cell medium (MEM medium with Earl's salts) containing 2 mM L-Glutamine, 0.4 mg/mL G418 Sulphate from GIBCO BRL and 10% heat inactivated fetal calf serum from Gibco BRL. The cells were seeded at 20,000 cells/100 μl/well into 96-well black clear bottom sterile plates from Costar which had been pre-coated with 10 μg/well of poly-L-lysine from SIGMA. The seeded plates were incubated overnight at 37° C. in 5% CO₂.

Agonists were prepared as 1 mM stocks in water:DMSO (1:1). EC₅₀ values (the concentration required to produce 50% maximal response) were estimated using 11× half log unit dilutions (Biomek 2000, Beckman) in Tyrode's buffer containing probenecid (10 mM HEPES with 145 mM NaCl, 10 mM glucose, 2.5 mM KCl, 1.5 mM CaCl₂, 1.2 mM MgCl₂ and 2.5 mM probenecid; pH7.4). Antagonists were prepared as 10 mM stocks in DMSO (100%). Antagonist IC₅₀ values (the concentration of compound needed to inhibit 50% of the agonist response) were determined against 3.0 nM human orexin-A using 11× half log unit dilutions in Tyrode's buffer containing 10% DMSO and probenecid.

On the day of assay 50 μl of cell medium containing probenecid (Sigma) and Fluo3AM (Texas Fluorescence Laboratories) was added (Quadra, Tomtec) to each well to give final concentrations of 2.5 mM and 4 μM, respectively. The 96-well plates were incubated for 90 min at 37° C. in 5% CO₂. The loading solution containing dye was then aspirated and cells were washed with 4×150 μl Tyrode's buffer containing probenecid and 0.1% gelatin (Denley Cell Wash). The volume of buffer left in each well was 125 μl. Antagonist or buffer (25 μl) was added (Quadra) the cell plates gently shaken and incubated at 37° C. in 5% CO₂ for 30 min. Cell plates were then transferred to the Fluorescent Imaging Plate Reader (FLIPR, Molecular Devices) instrument and maintained at 37° C. in humidified air. Prior to drug addition a single image of the cell plate was taken (signal test), to evaluate dye loading consistency. The run protocol used 60 images taken at 1 second intervals followed by a further 24 images at 5 second intervals. Agonists were added (by the FLIPR) after 20 sec (during continuous reading). From each well, peak fluorescence was determined over the whole assay period and the mean of readings 1-19 inclusive was subtracted from this figure. The peak increase in fluorescence was plotted against compound concentration and iteratively curve fitted using a four parameter logistic fit (as described by Bowen and Jerman, TiPS, 1995, 16, 413-417) to generate a concentration effect value. Antagonist Kb values were calculated using the equation:

K_{b}=IC₅₀/(1+([3/EC₅₀])

where EC₅₀ was the potency of human orexin-A determined in the assay (in nM terms) and IC₅₀ is expressed in molar terms.

Compounds of Examples tested according to this method had pKb values in the range 7.0-9.7 at the human cloned orexin-1 receptor.

The orexin-2 receptor antagonist activity of the compounds of formula (I) is determined in accordance with the following experimental method.

### Experimental Method

CHO-DG44 cells expressing the human orexin-2 receptor were grown in cell medium (MEM medium with Earl's salts) containing 2 mM L-Glutamine, 0.4 mg/mL G418 Sulphate from GIBCO BRL and 10% heat inactivated fetal calf serum from Gibco BRL. The cells were seeded at 20,000 cells/100 μl/well into 96-well black clear bottom sterile plates from Costar which had been pre-coated with 10 μg/well of poly-L-lysine from SIGMA. The seeded plates were incubated overnight at 37 C. in 5% CO₂.

Agonists were prepared as 1 mM stocks in water:DMSO (1:1). EC50 values (the concentration required to produce 50% maximal response) were estimated using 11× half log unit dilutions (Biomek 2000, Beckman) in Tyrode's buffer containing probenecid (10 mM HEPES with 145 mM NaCl, 10 mM glucose, 2.5 mM KCl, 1.5 mM CaCl₂, 1.2 mM MgCl₂ and 2.5 mM probenecid; pH7.4). Antagonists were prepared as 10 mM stocks in DMSO (100%). Antagonist IC50 values (the concentration of compound needed to inhibit 50% of the agonist response) were determined against 10.0 nM human orexin-A using 11× half log unit dilutions in Tyrode's buffer containing 10% DMSO and probenecid.

On the day of assay 50 μl of cell medium containing probenecid (Sigma) and Fluo3AM (Texas Fluorescence Laboratories) was added (Quadra, Tomtec) to each well to give final concentrations of 2.5 mM and 4 μM, respectively. The 96-well plates were incubated for 60 min at 37 C. in 5% CO₂. The loading solution containing dye was then aspirated and cells were washed with 4×150 μl Tyrode's buffer containing probenecid and 0.1% gelatin (Denley Cell Wash). The volume of buffer left in each well was 125 μl. Antagonist or buffer (25 μl) was added (Quadra) the cell plates gently shaken and incubated at 37 C. in 5% CO₂ for 30 min. Cell plates were then transferred to the Fluorescent Imaging Plate Reader (FLIPR, Molecular Devices) instrument. Prior to drug addition a single image of the cell plate was taken (signal test), to evaluate dye loading consistency. The run protocol used 60 images taken at 1 second intervals followed by a further 24 images at 5 second intervals. Agonists were added (by the FLIPR) after 20 sec (during continuous reading). From each well, peak fluorescence was determined over the whole assay period and the mean of readings 1-19 inclusive was subtracted from this figure. The peak increase in fluorescence was plotted against compound concentration and iteratively curve fitted using a four parameter logistic fit (as described by Bowen and Jerman, TiPS, 1995, 16, 413-417) to generate a concentration effect value. Antagonist Kb values were calculated using the equation:

Kb=IC50/(1+([3/EC50])

where EC50 was the potency of human orexin-A determined in the assay (in nM terms) and IC50 is expressed in molar terms.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1 A compound of formula (I): 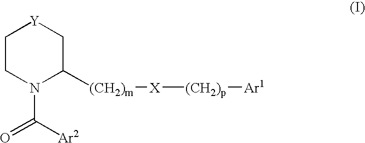
wherein:
Y represents a bond, oxygen, or a group (CH₂)ₙ, wherein n represents 1, 2 or 3
m represents 1, 2, or 3;
p represents 0 or 1;
X is O, S, C═O, SO₂, or —CH═CH—;
Ar¹ is aryl, or a mono or bicyclic heteroaryl group containing up to 4 heteroatoms selected from N, O and S; any of which may be optionally substituted;
Ar represents phenyl or a 5- or 6-membered heterocyclyl group containing up to 3 heteroatoms selected from N, O and S, wherein the phenyl or heterocyclyl group is substituted by R¹ and further optional substituents; or Ar² represents an optionally substituted bicyclic aromatic or bicyclic heteroaromatic group containing up to 3 heteroatoms selected from N, O and S;
R¹ represents hydrogen, optionally substituted(C₁₋₄ )alkoxy, halo, cyano, optionally substituted(C₁₋₆)alkyl, optionally substituted phenyl, or an optionally substituted 5- or 6-membered heterocyclic ring containing up to 4 heteroatoms selected from N, O and S;
or a pharmaceutically acceptable salt thereof:

2 A compound according to claim 1 wherein Y is a bond, oxygen or (CH₂)ₙ where n is 1 or 2.

3 A compound according to claim 1 wherein Ar² represents optionally substituted phenyl, pyridyl, thiazolyl, pyrazolyl, naphthyl, 1,2,3-triazolyl, thienyl or benzoxazolyl.

4 A compound according to claim 1 wherein R¹ represents a trifluoromethoxy group, methoxy group, ethoxy group, acetamido, halo, or an optionally substituted phenyl, pyridyl, pyrazolyl, pyrimidinyl, pyrazolyl or oxadiazolyl group.

5 A compound according to claim 1 wherein Ar¹ represents optionally substituted phenyl, naphthyl, pyridinyl or benzofuranyl.

6 A compound according claim 1 wherein Ar² is optionally substituted by halogen, cyano, (C₁₋₄)alkyl, hydroxy(C₁₋₄)alkyl, R³R⁴(CH₂)n, R³R⁴N, (C₁₋₄)alkanoyl or R³R⁴N(CH₂)nO.

7 A compound which is selected from:
2-(1-(2-Phenoxy)ethyl)-1-(2-phenyl)benzoylpiperidine;
2-(1-(2-(4-Chloro)phenoxy)ethyl)-1-(2-phenyl)benzoylpiperidine;
2-(1-(2-Phenoxy)ethyl)-1-(2-trifluoromethoxy)benzoylpiperidine;
2-(1-(2-Phenoxy)ethyl)-1-(2-(2-pyridyl))benzoylpiperidine;
2-(1-(2-Phenoxy)ethyl)-1-(2-(5-(3-methyl)-1,2,4-oxadiazolyl))benzoylpiperidine;
2-(1-(2-Phenoxy)ethyl)-1-(4-(2-methyl-5-phenyl)thiazolyl)carbonylpiperidine;
2-(1-(2-Phenoxy)ethyl)-1-(2-(4-fluoro)phenyl)benzoylpiperidine;
2-(1-(2-Phenoxy)ethyl)-1-(2-(2-cyano)phenyl)benzoylpiperidine;
2-(1-(2-Phenoxy)ethyl)-1-(2-(3-cyano)phenyl)benzoylpiperidine;
2-(1-(2-Phenoxy)ethyl)-1-(3-(2-phenyl)pyridyl)carbonylpiperidine;
2-(1-(2-(2-Cyano)phenoxy)ethyl)-1-(2-phenyl)benzoylpiperidine;
2-(1-(2-(3-Chloro)phenoxy)ethyl)-1-(2-phenyl)benzoylpiperidine;
2-(1-(2-(3,4-Dichloro)phenoxy)ethyl)-1-(2-phenyl)benzoylpiperidine;
2-(1-(2-Phenoxy)ethyl)-1-(2-(1-pyrazolyl))benzoylpiperidine;
2-(1-(2-(4-Fluoro)phenoxy)ethyl)-1-(2-phenyl)benzoylpiperidine;
2-(1-(2-(4-Fluoro)phenoxy)ethyl)-1-(2-(2-pyridyl))benzoylpiperidine;
2-(1-(2-(4-Fluoro)phenoxy)ethyl)-1-(2-(5-(3-methyl)-1,2,4-oxadiazolyl))benzoylpiperidine;
2-(1-(2-(4-Fluoro)phenoxy)ethyl)-1-(4-(2-methyl-5-phenyl)thiazolyl)carbonylpiperidine;
2-(1-(2-(3-Pyridyl)oxy)ethyl)-1-(4-(2-methyl-5-phenyl)thiazolyl)carbonylpiperidine;
2-(1-(2-(2-Pyridyl)oxy)ethyl)-1-(2-phenyl)benzoylpiperidine;
2-(1-(2-(2-Pyridyl)oxy)ethyl)-1-(2-(2-pyridyl))benzoylpiperidine;
2-(1-(2-(2-Pyridyl)oxy)ethyl)-1-(2-(5-(3-methyl)-1,2,4-oxadiazolyl))benzoylpiperidine;
2-(1-(2-(2-Pyridyl)oxy)ethyl)-1-(4-(2-methyl-5-phenyl)thiazolyl)carbonylpiperidine;
2-(1-(2-(1-Naphthyl)oxy)ethyl)-1-(2-(2-pyridyl))benzoylpiperidine;
2-(1-(2-(1-Naphthyl)oxy)ethyl)-1-(2-(5-(3-methyl)-1,2,4-oxadiazolyl))benzoylpiperidine;
2-(1-(2-(1-Naphthyl)oxy)ethyl)-1-(4-(2-methyl-5-phenyl)thiazolyl)carbonylpiperidine;
2-(1-(2-(1-Naphthyl)oxy)ethyl)-1-(2-trifluoromethoxy)benzoylpiperidine;
2-(1-(2-(1-Naphthyl)oxy)ethyl)-1-(1-naphthoyl)piperidine;
2-(1-(2-(3-Cyano)phenoxy)ethyl)-1-(2-phenyl)benzoylpiperidine;
2-(1-(2-(3-Cyano)phenoxy)ethyl)-1-(4-(2-methyl-5-phenyl)thiazolyl)carbonylpiperidine;
E:Z-2-(1-(2-(3-Phenyl)propenyl)-1-(4-(2-methyl-5-phenyl)thiazolyl)carbonylpiperidine;
1-(4-(2-Methyl-5-phenyl)thiazolyl)carbonyl)-2-(1-(2-phenoxy)ethyl)-pyrrolidine;
1-(4-(2-Methyl-5-phenyl)thiazolyl)carbonyl)-2-(1-(2-phenoxy)ethyl)-1H-2,3,4,5,6,7-hexahydroazepine;
4-(4-(2-Methyl-5-phenyl)thiazolyl)carbonyl)-3-(1-(2-phenoxy)ethyl)-morpholine;
3-(1-(2-(4-Fluorophenoxy))ethyl)-4-(4-(2-methyl-5-phenyl) thiazolyl)carbonyl)morpholine;
2-(1-(2-Phenoxy)ethyl)-1-(2-phenyl)benzoylpyrrolidine;
2-(1-(2-Phenoxy)ethyl)-1-(2-phenyl)benzoyl-1H-2,3,4,5,6,7-hexahydroazepine;
2-(1-(2-Phenoxy)ethyl)-1-(2-(2-pyridyl))benzoyl-1H-2,3,4,5,6,7-hexahydroazepine;
2-(1-(2-Phenoxy)ethyl)-1-(2-(5-(3-methyl)-1,2,4-oxadiazolyl))benzoyl-1H-2,3,4,5,6,7-hexahydroazepine;
2-(1-(2-Phenoxy)ethyl)-1-(2-trifluoromethoxy)benzoyl-1H-2,3,4,5,6,7-hexahydroazepine;
2-(1-(2-Phenoxy)ethyl)-1-(1-naphthoyl)-1H-2,3,4,5,6,7-hexahydroazepine;
3-(1-(2-Phenoxy)ethyl)-4-(2-phenyl)benzoylmorpholine;
2-(1-(2-Phenoxy)ethyl)-1-(5-fluoro-2-(5-(3-methyl)-1,2,4-oxadiazolyl))benzoylpiperidine;
1-(5-(3-Methyl-1-phenyl)-1H-pyrazolyl)carbonyl-2-(1-(2-phenoxy)ethyl)piperidine;
1-(4-(2-Methyl-5-phenyl)-2H-1,2,3-triazolyl)carbonyl-2-(1-(2-phenoxy)ethyl)piperidine;
1-(2-Iodo)benzoyl-2-(1-(2-phenoxy)ethyl)piperidine;
(R,S)-1-Benzofuran-2-yl-2-(1-{1-[5-(4-fluoro-phenyl)-2-methyl-thiazol-4-yl]-methanoyl}-piperidin-2-yl)-ethanone;
1-Benzofuran-2-yl-2-(1-{1-[4-(4-fluoro-phenyl)-1-H-pyrazol-3-yl]-methanoyl}piperidin-2-yl)-ethanone;
1-Benzofuran-2-yl-2-(1-{1-[4-(4-fluoro-phenyl)-1-methyl-1H-pyrazol-3-yl]-methanoyl}-piperidin-2-yl)-ethanone;
1-Benzofuran-2-yl-2-{1-[1-(5-bromo-2-methoxy-phenyl)-methanoyl]piperidin-2-yl}ethanone;
N(3-{1-[2-(2-Benzofuran-2-yl-2-oxo-ethyl)-piperidin-1-yl]-methanoyl}-phenyl)-acetamide;
1-(2-Benzyloxymethyl-piperidin-1-yl)-1-(2-methyl-5-phenyl-thiazol-4-yl)-methanone;
1-[2-(2-Benzyloxy-ethyl)-piperidin-1-yl]-1-(2-pyridin-2-yl-phenyl)-methanone;
1-[2-(2-Benzyloxy-ethyl)-piperidin-1-yl]-1-biphenyl-2-yl-methanone;
1-{2-[2-(4-Fluoro-benzyloxy)-ethyl]-piperidin-1-yl}-1-(2-methyl-5-phenyl-thiazol-4-yl)-methanone;
1-Biphenyl-2-yl-1-{2-[2-(4-fluoro-benzyloxy)-ethyl]-piperidin-1-yl}methanone;
1-(2-Methyl-5-phenyl-thiazol-4-yl)-1-[2-(3-phenoxy-propyl)-piperidin-1-yl]-methanone;
1-[5-(4-Fluoro-phenyl)-2-methyl-thiazol-4-yl]-1-{2-[2-(pyridin-2-ylsulphanyl)-ethyl]-piperidine-1-yl}-methanone;
3-[1-(1-Biphenyl-2-yl-methanoyl)-piperidine-2-yl]-1-phenyl-propan-1-one;
1-[(S)-2-(5-Bromo-pyrimidin-2-yloxymethyl)-pyrrolidin-1-yl]-1-[2-(3-methyl-[1,2,4]oxadiazol-5-yl)-phenyl]-methanone;
or a pharmaceutically acceptable salt thereof.

8 (Cancelled).

9 A pharmaceutical composition comprising a compound of formula (I) according to claim 1, or a pharmaceutically acceptable salt thereof, and a pharmaceutically acceptable carrier.

10 A method of treating or preventing diseases or disorders where an antagonist of a human orexin receptor is required, which comprises administering to a subject in need thereof an effective amount of a compound of formula (I) according to claim 1, or a pharmaceutically acceptable salt thereof.
